SEQUENCE LISTING (4 sequences)

SEQ ID NO: 1 - RNA, 19 nt, Artificial Sequence
  Synthetic Construct
ggacaaaccgccuuaauuc

SEQ ID NO: 2 - RNA, 19 nt, Artificial Sequence
  Synthetic Construct
cagcagaacuguaccuguu

SEQ ID NO: 3 - RNA, 19 nt, Artificial Sequence
  Synthetic Construct
gggcguggcuguccucaau

SEQ ID NO: 4 - RNA, 19 nt, Artificial Sequence
  Synthetic Construct
cgaaugaucacagcaauga

(19) United States
(12) Patent Application Publication
(10) Pub. No.: US 2023/0149346 A1
(43) Pub. Date: May 18, 2023

(54) 1,2,4-TRIOXANE COMPOUNDS AND COMPOSITIONS COMPRISING THE SAME FOR USE IN THE PREVENTION AND TREATMENT OF CANCER

(71) Applicants: Artemiflow GmbH, Potsdam (DE); University of Kentucky Research Foundation, Lexington, KY (US)

(72) Inventors: Adam Jeffrey MAUST, Dauphin, PA (US); Kerry GILMORE, Werder (DE); Peter SEEBERGER, Kleinmachnow (DE); Jill KOLESAR, Lexington, KY (US); Frederick UELAND, Lexington, KY (US); Kristen HILL, Lexington, KY (US); Anthony MCDOWELL, Lexington, KY (US)

(21) Appl. No.: 17/928,858

(22) PCT Filed: Jun. 2, 2021
(86) PCT No.: PCT/US2021/035524

§ 371 Date: Nov. 30, 2022

Related U.S. Application Data
(60) Provisional application No. 63/033,502, filed on Jun. 2, 2020.

(51) Int. Cl. A61K 31/357; A61K 31/216; A61K 36/282; A61K 36/82; A61K 36/74; A61K 31/555; A61P 35/04; A61K 31/337

(52) U.S. Cl. CPC A61K 31/357; A61K 31/216; A61K 36/282; A61K 36/82; A61K 36/74; A61K 31/555; A61P 35/04; A61K 31/337

(57) ABSTRACT

The present invention relates to anti-cancer agents comprising 1,2,4-trioxane compounds and anti-cancer agents comprising combinations comprising 1,2,4-trioxane compounds and chlorogenic acids. The invention further provides pharmaceutical compositions comprising such anti-cancer agents, kits comprising the same as well as methods and treatment regimens of using the aforementioned anti-cancer agents, pharmaceutical compositions and kits in the treatment and prevention of cancer and for prolonging survival of subjects having cancer, in particular ovarian cancer or lung cancer.

### CROSS REFERENCE TO RELATED APPLICATION

The application claims the benefit of U.S. Provisional Application No. 63/033,502 filed Jun. 2, 2021 and is incorporated by reference in its entirety herein.

### FIELD OF THE INVENTION

The present invention relates to anti-cancer agents comprising 1,2,4-trioxane compounds and anti-cancer agents comprising combinations comprising 1,2,4-trioxane compounds and chlorogenic acids. The invention further provides pharmaceutical compositions comprising such anti-cancer agents, kits comprising the same as well as methods and treatment regimens of using the aforementioned anti-cancer agents, pharmaceutical compositions and kits in the treatment and prevention of cancer and for prolonging survival of subjects having cancer, in particular ovarian cancer or lung cancer.

### BACKGROUND

Cancers remain a major health threat despite immense efforts in the search for cures. For example, ovarian cancer poses a major health problem in women worldwide and is the fourth leading cause of cancer death in women in the United States accounting for 5-6% of all cancer related mortality. The 5-year survival rate for early-stage patients is 80-90%, but only 40-50% for those diagnosed at advanced stages of the disease. Unfortunately, most ovarian cancer patients have advanced disease at diagnosis. Although the ovarian cancer mortality rate has not changed significantly during the past few decades, the length of survival for patients has been steadily improving, largely as a result of clinical applications of newer and more effective chemotherapeutic drugs for adjuvant therapy after surgery. For instance, paclitaxel and carboplatin are the most important chemotherapeutic drugs used for adjuvant treatment of primary ovarian cancer and for metastatic disease. Paclitaxel blocks cells in the G2/M phase of the cell cycle and such cells are unable to form a normal mitotic apparatus, carboplatin leads to formation of DNA adducts, resulting in G2 phase cell cycle arrest, subsequently triggering apoptosis. While effective, paclitaxel and carboplatin induce side effects, including myelosuppression, neurotoxicity and nephrotoxicity. Further, following completion of initial therapy, 70% of patients experience recurrence within the first two years after diagnosis, so that there is a pressing need for alternative or improved treatments.

Human hepatocellular carcinoma (HCC) is also one of the leading causes of cancer-related death worldwide, and more than 80% of liver cancer cases occur in developing countries, such as China and Africa. HCC has a long latency; therefore, it is often diagnosed at late stages when tumors are of high grade and progress rapidly. These characteristics, coupled with its high likelihood of invasion, lead to a poor prognosis for patients diagnosed with the disease. Nonsurgical approaches are necessary because patients with large tumors or numerous lesions typically are not suitable for hepatic resection. Unfortunately, the activity of single chemotherapeutic agents is limited, with a very low response rate. Aggressive combination chemotherapeutic regimens have not led to any remarkable improvement in response rates. In advanced HCC, cancer cells do not respond to the cytotoxic effects of most of the available chemotherapeutic agents. The same is true for some types of lung cancer, in particular non small cell lung cancer (NSCLC). Components of the kelch-like ECH-associated protein 1 (KEAP1)/nuclear factor erythroid 2-related factor 2 (NRF2) pathway, which regulates cellular response to oxidative stress, are mutated in approximately 30% of non-small-cell lung cancers (NSCLC). In NSCLC cell lines, overexpression of wild-type, but not inactivating mutant, KEAP1 results in reduced colony formation in soft agar, decreased cell migration, and reduced growth of tumors in subcutaneous xenografts. Additionally, overexpressing wild-type KEAP1 reduces the expression of NRF2 protein and the expression of transcriptional targets of NRF2 including heme oxygenase-1 (HO-1) and NAD(P)H quinone dehydrogenase 1 (NQO-1). Genetically engineered mouse models have also demonstrated that NRF2 activation or deletion of Keap1 accelerates Kras^{GI2D} driven lung tumorigenesis. When compared to individuals without mutations in this pathway, patients with mutations in the KEAP1/NRF2 pathway have significantly shorter progression-free survival and overall survival, less benefit from epidermal growth factor receptor (EGFR) inhibitors, insensitivity to chemotherapy, and increased metastasis. Inactivating KEAP1 mutations promote tumor growth and migration through activating NRF2 mediated transcription of antioxidant response genes like NQO1 and mutations in KEAP1/NRF2 are potential drivers of clinical treatment resistance to a variety of agents. Given the frequency and clinical significance of mutations in this pathway, effective treatment strategies for KEAP1/NRF2-mutated cancer are needed.

Various other cancers are in similar states as HCC, lung cancer such as non-small cell lung cancer (NSCLC) and ovarian cancer i.e. there has been some progress in the treatments or prevention; however, more effective treatments are still needed. Therefore, there remains a continued need for novel drugs that can be used alone or in combination with conventional agents.

Phytochemicals show promise in cancer therapy. Recently, several authors have demonstrated that the antimalarial drug artemisinin, its natural derivatives such as dihydroartemisisin and semisynthetic derivatives such as artesunate have the potential to be repurposed for use in anti-cancer regimens (see [1] Tsuda, K. et al. Mechanisms of the pH- and oxygen-dependent oxidation activities of artesunate. Biol Pharm Bull 41, 555-563 (2018); [2] Wang, B., Hou, D., Liu, Q., Wu, T., Guo, H., Zhang, X., Zou, Y., Liu, Z., Liu, J., Wei, J., Gong Y. & Shao, C. Artesunate sensitizes ovarian cancer cells to cisplatin by downregulating RAD51. Cancer Biology & Therapy 16, 1548-1556 (2015); [3] Chen, X., Wong, Y. K., Lim, T. K., Lim, W. H., Lin, Q., Wang, J. & Hua, Z. Artesunate activates the intrinsic apoptosis of HCT116 cells through the suppression of fatty acid synthesis and the NF-κB pathway. Molecules 22, 1272 (2017); [4] Kumar, B., Kalvala, A., Chu, S., Rosen, S., Forman, S. J., Marcucci, G., Chen, C. C. & Pullarkat, V. Antileukemic activity and cellular effects of the antimalarial agent artesunate in acute myeloid leukemia. Leuk Res 59, 124-135 (2017); [5] Liu, Y., Gao, S., Zhu, J., Zheng, Y., Zhang, H, & Sun H. Dihydroartemisinin induces apoptosis and inhibits proliferation, migration, and invasion in epithelial ovarian cancer via inhibition of the hedgehog signaling pathway. Cancer Med 7, 5704-5715 (2018); [6] Greenshields, A., Shepherd, T. & Hoskin, D. Contribution of reactive oxygen species to ovarian cancer cell growth arrest and killing by the anti-malarial drug artesunate. Molecular Carcinogenesis 56, 75-93 (2017), [7] Zhang, Z. Y.; Yu, S. Q.; Miao, L. Y.; Huang, X. Y.; Zhang, X. P.; Zhu, Y. P.; Xia, X. H.; Li, D. Q. Artesunate combined with vinorelbine plus cisplatin in treatment of advanced non-small cell lung cancer: A randomized controlled trial. Zhong Xi Yi Jie He Xue Bao 2008, 6, 134-138, doi:10.3736/jcim20080206; [8] Srinivas, U.S.; Tan, B. W. Q.; Vellayappan, B. A.; Jeyasekharan, A. D. ROS and the DNA damage response in cancer. Redox Biol. 2019, 25, 101084, doi:10.1016/j.redox.2018.101084; [9] Moloney, J. N.; Cotter, T. G. ROS signalling in the biology of cancer. Semin. Cell Dev. Biol. 2018, 80, 50-64, doi:10.1016/j.semcdb.2017.05.023, [10] Deeken, J. F.; Wang, H.; Hartley, M.; Cheema, A. K.; Smaglo, B.; Hwang, J. J.; He, A. R.; Weiner, L. M.; Marshall, J. L.; Giaccone, G.; et al. A phase I study of intravenous artesunate in patients with advanced solid tumor malignancies. Cancer Chemother. Pharmacol. 2018, 81, 587-596, doi:10.1007/s00280-018-3533-8, [11] Crespo-Ortiz, M. P., & Wei, M. Q. (2012). Antitumor activity of artemisinin and its derivatives: from a well-known antimalarial agent to a potential anticancer drug. J Biomed Biotechnol, 2012; [12] Efferth, T. (2017). From ancient herb to modern drug: Artemisia annua and artemisinin for cancer therapy. Semin Cancer Biol, 46, 65-83; Efferth, T., Sauerbrey, A., Olbrich, A., Gebhart, E., Rauch, P., Weber, H. O., Hengstler, J. G., Halatsch, M. E., Volm, M., Tew, K. D., Ross, D. D., & Funk, J. O. (2003). Molecular modes of action of artesunate in tumor cell lines. Mol Pharmacol, 64(2), 382-394; [13] Chen, H. H., Zhou, H. J., Wang, W. Q., & Wu, G. D. (2004). Antimalarial dihydroartemisinin also inhibits angiogenesis. Cancer Chemother Pharmacol, 53(5), 423-432; [14] Gao, P., Wang, L. L., Liu, J., Dong, F., Song, W., Liao, L., Wang, B., Zhang, W., Zhou, X., Xie, Q., Sun, R., & Liu, J. (2020). Dihydroartemisinin inhibits endothelial cell tube formation by suppression of the STAT3 signaling pathway. Life Sci, 242; [15] Konstat-Korzenny, E., Ascencio-Aragon, J. A., Niezen-Lugo, S., & Vazquez-Lopez, R. (2018). Artemisinin and Its Synthetic Derivatives as a Possible Therapy for Cancer. Med Sci (Basel), 6(1); [16] Slezakova, S., & Ruda-Kucerova, J. (2017). Anticancer Activity of Artemisinin and its Derivatives. Anticancer Res, 37(11), 5995-6003. https://doi.org/10.21873/anticanres.12046; [17] Tilaoui, M., Mouse, H. A., Jaafari, A., & Zyad, A. (2014). Differential effect of artemisinin against cancer cell lines. Nat Prod Bioprospect, 4(3), 189-196; [18] Deeken, J. F., Wang, H., Hartley, M., Cheema, A. K., Smaglo, B., Hwang, J. J., He, A. R., Weiner, L. M., Marshall, J. L., Giaccone, G., Liu, S., Luecht, J., Spiegel, J. Y., & Pishvaian, M. J. (2018). A phase I study of intravenous artesunate in patients with advanced solid tumor malignancies. Cancer Chemother Pharmacol, 81(3), 587-596; [19] Jiao, Y., Ge, C. M., Meng, Q. H., Cao, J. P., Tong, J., & Fan, S. J. (2007). Dihydroartemisinin is an inhibitor of ovarian cancer cell growth. Acta Pharmacol Sin, 28(7), 1045-1056; [20] Sertel, S., Eichhorn, T., Simon, C. H., Plinkert, P. K., Johnson, S. W., & Efferth, T. (2010). Pharmacogenomic identification of c-Myc/Max-regulated genes associated with cytotoxicity of artesunate towards human colon, ovarian and lung cancer cell lines. Molecules, 15(4), 2886-2910; [21] von Hagens, C., Walter-Sack, I., Goeckenjan, M., Storch-Hagenlocher, B., Sertel, S., Elsasser, M., Remppis, B. A., Munzinger, J., Edler, L., Efferth, T., Schneeweiss, A., & Strowitzki, T. (2019). Long-term add-on therapy (compassionate use) with oral artesunate in patients with metastatic breast cancer after participating in a phase I study (ARTIC M33/2). Phytomedicine, 54, 140-148; [22] Konig, M., von Hagens, C., Hoth, S., Baumann, I., Walter-Sack, I., Edler, L., & Sertel, S. (2016). Investigation of ototoxicity of artesunate as add-on therapy in patients with metastatic or locally advanced breast cancer: new audiological results from a prospective, open, uncontrolled, monocentric phase I study. Cancer Chemother Pharmacol, 77(2), 413-427; [23] Li, Q., Ni, W., Deng, Z., Liu, M., She, L., & Xie, Q. (2017). Targeting nasopharyngeal carcinoma by artesunate through inhibiting Akt/mTOR and inducing oxidative stress. Fundam Clin Pharmacol, 31(3), 301-310; [24] Li, Y., Shan, F., Wu, J. M., Wu, G. S., Ding, J., Xiao, D., Yang, W. Y., Atassi, G., Leonce, S., Caignard, D. H., & Renard, P. (2001). Novel antitumor artemisinin derivatives targeting G1 phase of the cell cycle. Bioorg Med Chem Lett, 11(1), 5-8; [25] Liu, L., Zuo, L. F., Zuo, J., & Wang, J. (2015). Artesunate induces apoptosis and inhibits growth of Eca109 and Ec9706 human esophageal cancer cell lines in vitro and in vivo. Mol Med Rep, 12(1), 1465-1472; [26] Luo, J., Zhu, W., Tang, Y., Cao, H., Zhou, Y., Ji, R., Zhou, X., Lu, Z., Yang, H., Zhang, S., & Cao, J. (2014). Artemisinin derivative artesunate induces radiosensitivity in cervical cancer cells in vitro and in vivo. Radiat Oncol, 9, 84; [27] Morrissey, C., Gallis, B., Solazzi, J. W., Kim, B. J., Gulati, R., Vakar-Lopez, F., Goodlett, D. R., Vessella, R. L., & Sasaki, T. (2010). Effect of artemisinin derivatives on apoptosis and cell cycle in prostate cancer cells. Anticancer Drugs, 21(4), 423-432; [28] Roh, J. L., Kim, E. H., Jang, H., & Shin, D. (2017). Nrf2 inhibition reverses the resistance of cisplatin-resistant head and neck cancer cells to artesunate-induced ferroptosis. Redox Biol, 11, 254-262; [29] Sertel, S., Eichhorn, T., Sieber, S., Sauer, A., Weiss, J., Plinkert, P. K., & Efferth, T. (2010). Factors determining sensitivity or resistance of tumor cell lines towards artesunate. Chem Biol Interact, 185(1), 42-52; [30] Zhang, J., Sun, X., Wang, L., Wong, Y. K., Lee, Y. M., Zhou, C., Wu, G., Zhao, T., Yang, L., Lu, L., Zhong, J., Huang, D., & Wang, J. (2018). Artesunate-induced mitophagy alters cellular redox status. Redox Biol, 19, 263-273).

Further literature can be found in: [31] McDowell, A., Jr.; Hill, K. S.; McCorkle, J. R.; Gorski, J.; Zhang, Y.; Salahudeen, A.; Ueland, F.; Kolesar, J. M., “Preclinical Evaluation of Artesunate as an Antineoplastic Agent in Ovarian Cancer Treatment”, Diagnostics 2021, 11, 395 and [32] Hill, K. S.; McDowell, A.; McCorkle, R.; Schuler, E.; Ellingson, S.; Plattner, R.; Kolesar, J. M. “KEAP1 is Required for Artesunate Anticancer Activity in Non-Small-Cell Lung Cancer”, Cancers 2021, 13, 1885.

Even though many mechanisms such as induction of apoptosis, inhibition of angiogenesis, inhibition of hypoxia-inducible factor-1a (HIF-1a) activation and direct DNA injury were proposed, the commonly assumed primary mode of action is the generation of reactive oxygen species (ROS) in both the cytoplasm and the mitochondria and mitochondria dependent apoptosis.

However, while encouraging results were obtained there's still a further need to understand the mode of action in order to improve cancer treatments and prevention in particular for cancers with still high mortality such as ovarian, liver and lung cancers.

### SUMMARY OF THE INVENTION

The present invention relates to anti-cancer agents comprising at least one compound having at least one 1,2,4-trioxane moiety or a combination of
a) at least one compound having at least one 1,2,4-trioxane moiety and
b) at least one chlorogenic acid.

The combinations exhibit even more potent activity against cancer cells in vitro compared to compounds having at least one 1,2,4-trioxane moiety such artemisinin, and its natural or synthetic derivatives alone. They further have a very good safety profile and thus provide an opportunity for a broad clinical use without severe side effects.

The invention further provides pharmaceutical compositions comprising such anti-cancer agents as well as methods to use the aforementioned anti-cancer agents andpharmaceutical compositions to prevent or treat cancer or for prolonging survival of subjects having cancer including delaying or preventing recurrence of cancer. Further objects of this invention are described herein below.

In another aspect the invention relates to kits comprising the anti-cancer agents or pharmaceutical compositions according to the invention as well as at least one additional therapeutic agent.

### BRIEF DESCRIPTION OF THE DRAWINGS

FIG. 1 shows Artesunate sensitivities across 3 commercially available ovarian cancer cell lines (Caov-3, OVCAR-3, and UWB1.289) whereby cells were treated with serially diluted concentrations of artesunate for 72 h. CellTiter-Glo 2.0 viability assay (Promega) was used to calculate percent viability from the proliferation of treated versus vehicle treated control cells. The mean+/− SD from three independent experiments is shown graphically and IC50s were calculated using a variable slope non-linear regression line.

FIG. 2 shows a DNA damage assay as average nuclear pH2AX intensity. The average nuclear intensity of pH2AX staining was quantified in Caov-3 cells treated for 48 h with artesunate concentrations ranging from 5-100 μM with 25 μM cisplatin treatment as a positive control. The mean signal+/− SD was graphed and a one-tailed t-test was performed (* p=0.0486, ** p=0.0034).

FIGS. 3 to 6 show a cell cycle analysis using propidium iodide staining. The percentage of Caov-3 and UWB1 cells in G1 after 24 h (FIG. 3) or 48 h (FIG. 4) treatment with 0.1% DMSO (control) or 10 μM artesunate is graphed as the mean+/− SD from three independent experiments. A one-tailed unpaired t-test revealed a statistically significant increase in cells in G1 in UWB1 cells treated for both 24 or 48 h (* p<0.05) and in Caov-3 cells treated with artesunate for 48 h (** p<0.01). The concurrent analysis of the percentage of cells in S-phase following 24 (FIG. 5) or 48 (FIG. 6) hour treatment with artesunate reveals a significant decrease in cells in S-phase after 48 h (* p<0.05; *** p<0.001).

FIGS. 7 and 8 show an anti-cancer agents drug administration sequence assay for artesunate, carboplatin, and paclitaxel. Cells were treated with artesunate on day 1 (D1A) or day 2 (D2A), carboplatin and paclitaxel on day 2 (D2C/T), carboplatin, paclitaxel, and artesunate on day 2 (D2C/T/A), or artesunate on day 1 followed by carboplatin and paclitaxel on day 2 (D1A; D2C/T). The 24 h treatment concentrations for artesunate, carboplatin, and paclitaxel were 40 μM, 16 μM, and 32 μM, respectively. Percent viability was calculated utilizing the CellTiter-Glo 2.0 viability assay following treatment with carboplatin/paclitaxel and/or artesunate compared to DMSO (control) treated cells, as indicated, shown graphically as the mean+/— SD in Caov-3 (FIG. 7) and UWB1 (FIG. 8) cells. Statistical differences were assessed by One-way ANOVA (ns—not significant and * p<0.05). In both cell lines, the addition of artesunate as a concurrent treatment with carboplatin/paclitaxel resulted in a significant decrease in viable cells.

FIGS. 9 and 10 show Artesunate sensitivity across a panel of non-small-cell lung cancer (NSCLC) cell lines. Cells were treated with a serially diluted concentrations of artesunate for 96 hr. Each cell lines was normalized to cells treated with 0.1% DMSO (dimethyl sulfoxide) as a control and is graphed as the mean±SD, n=6 (FIG. 9). The mean IC50 of artesunate (Art) in each cell line is graphed ±SD. p-values were calculated using a two-tailed t-test (* p=0.0290 or ** p=0.0007 compared to A549) (FIG. 10).

FIG. 11 shows a dose-dependent induction of DNA damage by artesunate in NSCLC cell lines quantified by nuclear pH2AX staining. Graphed as mean fold change in nuclear fluorescent intensity signal normalized to 0.1% DMSO per cell ±SD. p-values calculate by one-way ANOVA for each cell line with Dunnett's multiple comparison test comparing to matched 0.1% DMSO control (* p<0.05, *** p<0.001).

FIG. 12 shows that Artesunate treatment induces changes in kelch-like ECH-associated protein 1 (KEAP1)/nuclear factor erythroid 2-related factor 2 (NRF2) pathway protein expression in a time-dependent manor in A549 and H1299 NSCLC cell lines. Cells were treated with 10 μM artesunate for 0, 6, or 24 h and levels of KEAP1 and NQO-1 (NAD(P)H quinone dehydrogenase 1) proteins were assessed by Western blot. In both cell lines, KEAP1 protein levels are reduced following treatment with artesunate.

FIG. 13 shows a Western blot analysis for KEAP1, NQO-1 and β-Actin and reduced KEAP1 protein in siKEAP1-treated cells 48 h after transfection.

FIG. 14 shows Artesunate dose-response curves after transfection with non-targeting (siNT—solid) or KEAP1 (siKEAP1—dashed) siRNA. Data was normalized to 0.1% DMSO and is plotted as mean±SD, n=4.

FIG. 15 shows the mean IC50 of artesunate in each cell line transfected with non-targeting (siNT) siRNA (solid) or siKEAP1 (open) ±SD. p-values were calculated using a two-tailed t-test.

FIGS. 16 and 17 show the quantification of nuclear pH2AX staining in A549 (FIG. 16) and H1299 (FIG. 17) following transfection with siNT or siKEAP1. DNA damage was assessed by nuclear pH2AX staining after 24 h treatment with 0.1% DMSO (control), the indicated concentration of artesunate, or 25 μM cisplatin. Nuclear pH2AX staining is was normalized to cells transfected with siNT and treated with DMSO control and is plotted as mean fold change ±SD. p-values calculate using a two-way ANOVA comparing each artesunate concertation to the matched control after normalization (* p<0.05; *** p<0.001).

FIG. 18 shows that pharmacological inhibition of NRF2 sensitizes A549 cells to artesunate, whereby Artesunate was added to media with 0.1% DMSO (solid) or 5 μM ML385 (dashed) and artesunate dose response data was normalized to DMSO or 5 μM ML385 alone and is graphed as mean±SD, n=6.

FIG. 19 graphs the mean IC50 of artesunate in each cell line with 0.1% DMSO (solid) or 5 μM ML385 (open) ±SD. p-values were calculated using a two-tailed t-test.

FIGS. 20 and 21 show the quantification of nuclear pH2AX staining in A549 (FIG. 20) and H1299 (FIG. 21) cells that were pretreated for 24 h with 0.03% DMSO or 5 μM ML385 followed by the addition of the indicated concentration of artesunate for an additional 24 h. Graphed as mean fold change ±SD in nuclear fluorescent intensity signal normalized to vehicle control cells pretreated with 0.03%. P-values calculate using a one-way ANOVA with Dunnett's multiple comparison test comparing each artesunate concertation to the matched control after normalization (** p<0.01; *** p<0.001).

FIGS. 22 and 23 show a_graphic representation of the Bliss independence model of synergy scoring as calculated by using a 6×6 dose-response matrix in A549 (FIG. 22) and H1299 (FIG. 23) cells. Red color indicates synergy, while green indicates antagonism between the drug combinations tested.

### DETAILED DESCRIPTION

For purposes of interpreting this specification, the following definitions will apply, and whenever appropriate, terms used in the singular will also include the plural.

Terms used in the specification have the following meanings unless the context clearly indicates otherwise:

As used herein, the term “cancer” means a group of diseases involving abnormal cell growth with the potential to invade or spread to other parts of the body and includes melanoma; multiple myeloma; carcinoma such as adenocarcinoma e.g. of the breast, colon, and prostate, basal cell carcinoma, squamous cell carcinoma, transitional cell carcinoma; Sarcoma such as osteosarcoma, leiomyosarcoma, kaposi sarcoma, malignant fibrous histiocytoma, liposarcoma, and dermatofibrosarcoma protuberans; leukemia such as acute lymphoblastic leukemia, acute myeloid leukemia, chronic myelogenous leukemia, chronic lymphocytic leukemia and hairy cell leukemia; lymphoma such as Hodgkin's lymphoma and non-Hodgkin's lymphoma; brain and spinal cord tumors, germ cell tumors, neuroendocrine tumors and carcinoid tumors.

The aformentioned types of cancer include the following types of cancer also referred to in literature: colorectal cancer, breast cancer, lung cancer, ovarian cancer, cervical cancer, bladder cancer, prostate cancer, gastric cancer, liver cancer, pancreatic cancer and thyroid cancer.

Preferred cancers treated by the compositions according to the invention include lung cancer, in particular non small cell lung cancer (NSCLC), ovarian cancer, and liver cancer.

As used herein, the term “subject” refers to an animal. In certain aspects, the animal is a mammal. A subject also refers to for example, primates (e.g. humans), cows, sheep, goats, horses, dogs, cats, poultry, rabbits, rats, mice, fish, birds and the like. In certain embodiments, the subject is a human. A “patient” as used herein refers to a human subject.

As used herein, the term “inhibition” or “inhibiting” refers to the reduction or suppression of a given condition, symptom, or disorder, or disease in particular cancer, or a significant decrease in the baseline activity of a biological activity or process.

As used herein the term “prolonging survival” means extending the life span of a subject having cancer by at least one day versus a subject having the same cancer that does not receive the anti-cancer agents, pharmaceutical compositions or kits according to the present invention.

Prolonged survival includes increasing the life span of the subject by at least: 1, 2, 3, 4 or more weeks, 1, 2, 3, 4, 5, 6, 7, 8, 9, 10, 11, 12 or more months, or 1, 2, 3, 4, 5 or more years.

As used herein, the term “treating” or “treatment” of cancer refers in one embodiment, to ameliorating the cancer i.e. slowing or arresting or reducing the development of cancer at least one of the clinical symptoms thereof. In another embodiment “treating” or “treatment” refers to alleviating or ameliorating at least one physical parameter including those which may not be discernible by the patient. In yet another embodiment. “treating” or “treatment” refers to modulating cancer, either physically, e.g. stabilization of a discernible symptom, physiologically, e.g., stabilization of a physical parameter, or both. In yet another embodiment, “treating” or “treatment” refers to preventing or delaying the onset, development, progression or recurrence of cancer.

As used herein, the terms “administered”, “administration”, “co-administered” and “co-administration” refer to administering to the subject the combination of compounds contemplated herein and optionally along with at least one additional compound that may also treat cancer as described herein.

In one embodiment, the compounds are administered separately as part of a single therapeutic approach in any specific sequence. In a preferred embodiment, the compounds of the combination according to the invention are co-administered in a joint formulation e.g. as a pharmaceutical composition according to the invention.

As used herein, the term “a,” “an,” “the” and similar terms used in the context of the present invention especially in the context of the claims are to be construed to cover both the singular and plural unless otherwise indicated herein or explicitly contradicted by the context.

All methods described herein can be performed in any suitable order unless otherwise indicated herein or otherwise clearly contradicted by context. The use of any and all examples, or exemplary language e.g. “such as” provided herein is intended merely to better illustrate the invention and does not pose a limitation on the scope of the invention otherwise claimed.

“Optionally substituted” or “substituted” means one or more hydrogen atoms at any position in the molecule or moiety referred to can be substituted by any one or any combination of substituents with their number, placement and selection being understood to encompass only those substitutions that a skilled chemist would expect to be reasonably stable.

Various embodiments of the invention are described herein. It will be recognized that features specified in each embodiment may be combined with other specified features to provide further embodiments.

The combinations according to the invention comprise at least one compound comprising at least one 1,2,4-trioxane moiety, such compounds being those comprising at least one 1,2,4-trioxane ring which is optionally, but preferably substituted.

In one embodiment the compounds comprising at least one 1,2,4-trioxane moiety are selected from those of formulae (I) to (V)

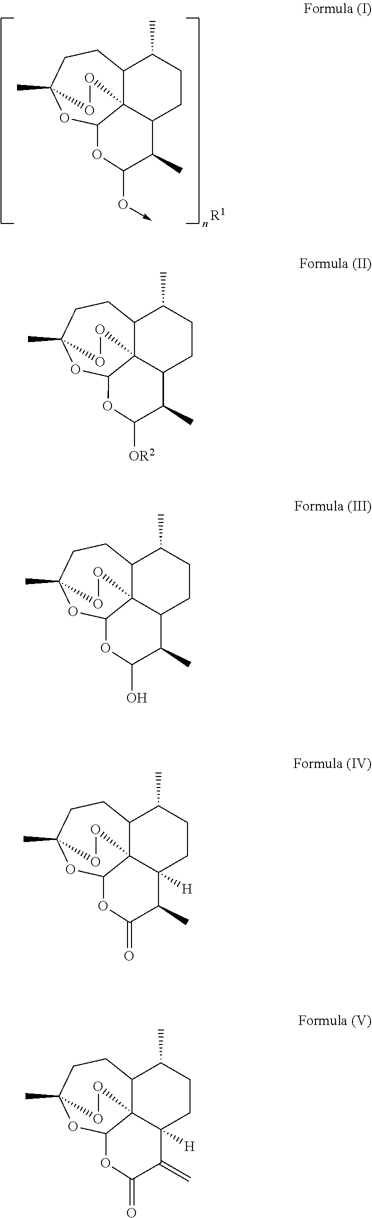

and, where applicable, pharmaceutically acceptable salts of the aforementioned compounds of formulae (I) and (II).

In formula (I)

the arrow denotes the bond between the depicted oxygen atom to the residue R¹
n is an integer of more than 1, preferably 2 to 10, more preferably 2, 3 or 4 and even more preferably 2 or 3
R¹ is a residue that is n times substitued by the residue depicted in the rounded bracket, and is preferably C₁-C₁₈-alkyl or C₂-C₁₈-alkenyl or —(CO)ₙ(R³), wherein the carboyl groups together with the oxygen bound to the residue R¹ form a carboxylic ester moiety and R³ is C₁-C₁₈-alkane-n-yl or C₂-C₁₈-alkene-n-yl

whereby

the aforementioned C₁-C₁₈-alkyl, C₂-C₁₈-alkenyl, C₂-C₁₈-alkane-n-yl, C₂-C₁₈-alkene-n-yl groups are
either not, once, twice, or more than twice interrupted by non-successive functional groups selected from the group consisting of:
   —O—, —S—, —SO₂—, —SO—, —SO₂NR⁴—, NR⁴SO₂—, —NR⁴—, —CO—, —O(CO)—, (CO)O—, —O(CO)O—, —NR⁴(CO)NR⁴—, NR⁴(CO)—, —(CO)NR⁴—, —NR⁴(CO)O—, —O(CO)NR⁴—,
and
either not, additionally, or alternatively either once, twice or more than twice interrupted by bivalent residues selected from the group consisting of heterocyclo-diyl, and aryldiyl,
and
either not, additionally, or alternatively either once, twice or more than twice substituted by substituents selected from the group consisting of:
   hydroxy, halogen, cyano, azido, C₆-C₁₄-aryl, C₁-C₈-alkoxy, C₁-C₈-alkylthio, —SO₃M, —COOM, PO₃M₂, —PO(N(R⁵)₂)₂, PO(OR⁵)₂, —SO₂N(R⁴)₂, —N(R⁴)₂, —CO₂N(R⁵)₂, —COR⁴, —OCOR⁴, —NR⁴(CO)R⁵, —(CO)OR⁴, —NR⁴(CO)N(R⁴)₂.

In formula (II)
R² is C₁-C₁₈-alkyl or C₂-C₁₈-alkenyl or —(CO)R³, wherein the carboyl groups together with the oxygen bound to the residue R¹ form a carboxylic ester moiety and R³ is C₁-C₁₈-alkyl or C₂-C₁₈-alkenyl whereby
   the aforementioned C₁-C₁₈-alkyl and C₂-C₁₈-alkenyl groups are
      either not, once, twice, or more than twice interrupted by non-successive functional groups selected from the group consisting of:
         —O—, —S—, —SO₂—, —SO—, —SO₂NR⁴—, NR⁴SO₂—, —NR⁴—, —CO—, —O(CO)—, (CO)O—, —O(CO)O—, —NR⁴(CO)NR⁴—, NR⁴(CO)—, —(CO)NR⁴—, —NR⁴(CO)O— or —O(CO)NR⁴—
   and
      either not, additionally, or alternatively either once, twice or more than twice interrupted by bivalent residues selected from the group consisting of heterocyclo-diyl, and aryldiyl,
   and
      either not, additionally, or alternatively either once, twice or more than twice substituted by substituents selected from the group consisting of:
      hydroxy, halogen, cyano, azido, C₆-C₁₄-aryl, C₁-C₈-alkoxy, C₁-C₈-alkylthio, —SO₃M, —COOM, PO₃M₂, —PO(N(R⁵)₂)₂, PO(OR⁵)₂, —SO₂N(R⁴)₂, —N(R⁴)₂, —CO₂N(R⁵)₂, —COR⁴, —OCOR⁴, —NR⁴(CO)R⁵, —(CO)OR⁴ or —NR⁴(CO)N(R⁴)₂

whereby in all formulae above where used
R⁴ is independently selected from the group consisting of hydrogen, C₁-C₈-alkyl, C₆-C₁₄-aryl, and heterocyclyl or N(R⁴)₂ as a whole is a N-containing heterocycle,
R⁵ is independently selected from the group consisting of C₁-C₈-alkyl, C₆-C₁₄-aryl, and heterocyclyl or N(R⁵)₂ as a whole is a N-containing heterocycle and
M is hydrogen, or 1/q equivalent of an q-valent metal ion or is an ammonium ion or a guanidinium ion or a primary, secondary, tertiary or quarternary organic ammonium ion, in particular those of formula [N(C₁-C₁₈-alkyl)ₛHₜ]⁺ wherein s is 1,2,3 or 4 and t is (4-s).

As used herein, and unless specifically stated otherwise, C₁-C₁₈-alkyl, C₁-C₁₈-alkene-n-yl, C₁-C₈-alkyl, C₁-C₈-alkoxy and C₁-C₈-alkylthio include straight-chained or, for C₃-C₁₈ or C₃-C₈ also cyclic either in part or as a whole, branched or unbranched alkyl, alkoxy, and alkylthio substituents having the given number of carbon atoms in the substituent as such.

As used herein, and unless specifically stated otherwise, C₂-C₁₈-alkenyl include straight-chained or, for C₅-C₁₈ also cyclic either in part or as a whole, branched or unbranched alkenyl, having the given number of carbon atoms in the substituent as such.

As used herein, and unless specifically stated otherwise, C₆-C₁₄-aryl, C₆-C₁₄-aryloxy, and C₆-C₁₄-arylthio denote carbocyclic aromatic substituents having six to fourteen carbon atoms within the aromatic system as such, i.e. without carbon atoms of substituents, preferably phenyl (C₆), naphthyl (C₁₀), phenanthrenyl and anthracenyl (each C₁₄), whereby said carbocyclic, aromatic substituents are either unsubstituted or substituted by up to five identical or different substituents per cycle. For example and with preference, the substituents are selected from the group consisting of fluoro, chloro, C₁-C₁₈-alkyl, C₁-C₁₈-alkoxy, C₆-C₁₄-aryl.

In a more preferred embodiment the carbocyclic, aromatic substituents are unsubstituted.

Specific examples of C₁-C₁₈-alkyl are methyl, ethyl, n-propyl, isopropyl, n-butyl, sec-butyl, tert-butyl, n-pentyl, isopentyl, tert.-pentyl, neopentyl, cyclohexyl, n-hexyl, n-heptyl, n-octyl and isooctyl, n-decyl, n-dodecyl n-hexadecyl, n-octadecyl.

Specific examples of C₁-C₈-alkoxy-substituents are methoxy, ethoxy, isopropoxy, n-propoxy, n-butoxy, sec.-butoxy, tert-butoxy and cyclohexyloxy.

Specific examples of C₁-C₈-alkylthio-substituents are methylthio and ethylthio.

Specific examples of C₆-C₁₄-aryl are phenyl, o-, m-, and p-tolyl.

A further specific example of an C₆-C₁₄-aryl-substituent is phenoxy.

A further specific example of an C₆-C₁₄-aryl-substituent is phenylthio.

Preferred compounds of formula (II) are those of formula (IIa), artemether, and of formula (IIb), artesunate, and pharmaceutically acceptable salts of artesunate.

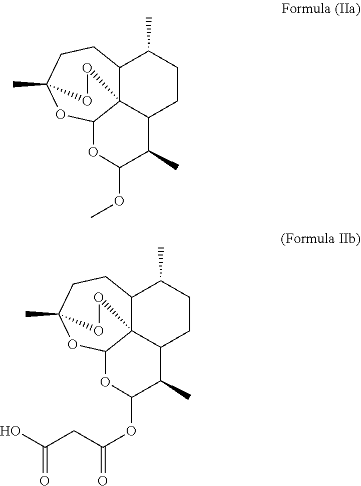

The compound of formula (III) is dihydroartemisin.

The compound of formula (IV) is artemisinin.

The compound of formula (V) is artemisitene.

In one embodiment of the invention the anti-cancer agents according to the invention contain more than one compound comprising at least one 1,2,4-trioxane moiety and preferably more than one compound selected from those of formulae (I) to (V) above and, where applicable, pharmaceutically acceptable salts of such compounds.

In one preferred embodiment the combination according to the invention comprises at least two, for example two, three, four or all the compounds selected from formula (IIa), (IIb), (III), (IV) and (V).

Naturally occurring compounds comprising at least one 1,2,4-trioxane moiety and combinations of such compounds may be employed for the purposes of this invention by using the plant Artemisia annua or parts thereof as such or may be obtained via known extraction methods from Artemisia annua as and, where desired, standard workup methods e.g. as published in Triemer et al., Angewandte Chemie, International Edition 57, (2018), p. 5525-5528.

Where Artemisia annua is extracted, this may occur using the whole plant or parts thereof such as leaves or stems, whether dried or freshly harvested. Suitable solvents for extraction include hexanes, cyclohexane, supercritical carbon dioxide, hydrofluorocarbon HFC-134a, ionic liquids, water, methanol, ethanol, 1-butanol, acetone, cyclohexanone, toluene, ethyl acetate, acetonitrile, tetrahydrofuran, or mixtures thereof.

Where e.g. single compounds comprising at least one 1,2,4-trioxane moiety of the invention are desired to be used in the combination extracts of Artemisia annua can be separated in a manner known per se to obtain the individual compounds for example, by partitioning between polyphasic solvent mixtures, recrystallization and/or chromatographic separation, for example over silica gel or by, e.g., medium pressure liquid chromatography over a reversed phase column or by fractional crystallization.

In one embodiment the Artemisia annua plant is of the Apollon variety, see X. Simmonet et al., “Apollon, a new Artemisia annua variety with high artemisinin content”, Planta Medica, 2011, 77(12) which is commercially available from the company Mediplant, Conthey Switzerland.

The individual compounds comprising at least one 1,2,4-trioxane moiety can be worked up and/or purified according to standard methods, e.g., using chromatographic methods, distribution methods, (re-) crystallization, and the like.

Synthetic or semi-synthetic compounds comprising at least one 1,2,4-trioxane moiety are for example prepared by preparation methods known to those skilled in the art and some of which are published e.g. in Reiter, C., Fröhlich, T., Gruber, L., Hutterer, C., Marschall, M., Voigtländer, C., Friedrich, O., Kappes, B., Efferth, T., Tsogoeva, S. B., 2015a. Highly potent artemisinin-derived dimers and trimers: Synthesis and evaluation of their antimalarial, antileukemia and antiviral activities. Bioorg. Med. Chem. 23 (17), 5452-5458; Reiter, C., Fröhlich, T., Zeino, M., Marschall, M., Bahsi, H., Leidenberger, M., Friedrich, O., Kappes, B., Hampel, F., Efferth, T., Tsogoeva, S. B., 2015b. New efficient artemisinin derived agents against human leukemia cells, human cytomegalovirus and Plasmodium falciparum: 2nd generation 1,2,4-trioxane-ferrocene hybrids. Eur. J. Med. Chem. 97, 164-172; Posner, G. H., Ploypradith, P., Parker, M. H., O'Dowd, H., Woo, S. H., Northrop, J., Krasavin, M., Dolan, P., Kensler, T. W., Xie, S., Shapiro, T. A., 1999. Antimalarial, antiproliferative, and antitumor activities of artemisinin-derived, chemically robust, trioxane dimers. J. Med. Chem. 42 (21), 4275-4280; Paik, I. H., Xie, S., Shapiro, T. A., Labonte, T., Narducci Sarjeant, A. A., Baege, A. C., Posner, G. H., 2006. Second generation, orally active, antimalarial, artemisinin-derived trioxane dimers with high stability, efficacy, and anticancer activity. J. Med. Chem. 49 (9), 2731-2734, Li, Y., Zhu, Y. M., Jiang, H. J., Pan, J. P., Wu, G. S., Wu, J. M., Shi, Y. L., Yang, J. D., Wu, B. A., 2000. Synthesis and antimalarial activity of artemisinin derivatives containing an amino group. J. Med. Chem. 43 (8), 1635-1640; Ren, Y., Yu, J., Kinghorn, A. D., 2016. Development of anticancer agents from plant-derived sesquiterpene lactones. Curr. Med. Chem. 23 (23), 2397-2420. O'Neill, P. M., Searle, N. L., Kan, K. W., Storr, R. C., Maggs, J. L., Ward, S. A., Raynes, K., Park, B. K., 1999. Novel, potent, semisynthetic antimalarial carba analogues of the first-generation 1,2,4-trioxane artemether. J. Med. Chem. 42 (26), 5487-5493 which are hereby incorporated by reference.

The anti-cancer agents according to the invention may comprise a combination of at least one compound having at least one 1,2,4-trioxane moiety and at least one chlorogenic acid.

As used herein the term “chlorogenic acid” or chlorogenic acids” denote compounds wherein one or two hydroxyl groups of quinic acid are esterified with caffeic, ferulic or p-coumaric acid.

Preferred examples of chlorogenic acids include 3-O-caffeoylquinic acid (formula VI a), 4-O-caffeoylquinic acid (formula VI b), 5-O-caffeoylquinic acid (formula VI c), 3-O-ferruoylquinic acid (formula VI d), 4-O-ferruoylquinic acid (formula VI e), 5-O-ferruoylquinic acid (formula VI f), 3,4-dicaffeoylquinic acid (formula VII a), 3,5-dicaffeoylquinic acid (formula VII b) and 4,5-dicaffeoylquinic acid (formula VII c), whereby 3-O-caffeoylquinic acid (formula VI a), 4-O-caffeoylquinic acid (formula VI b), 5-O-caffeoylquinic acid (formula VI c), 3,4-dicaffeoylquinic acid (formula VII a), 3,5-dicaffeoylquinic acid (formula VII b) and 4,5-dicaffeoylquinic acid (formula VII c) are even more preferred.

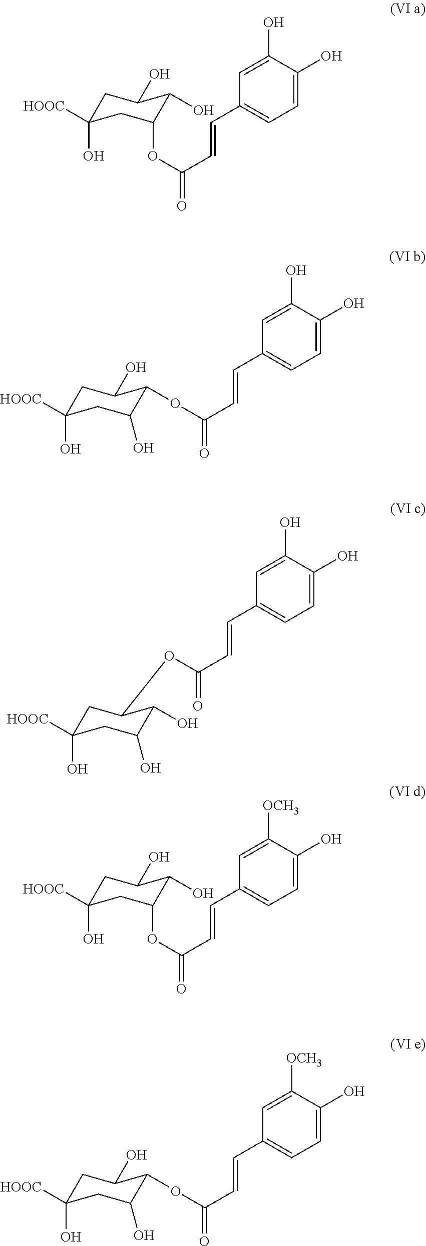 [SMILES: O=C(/C=C/c1ccc(O)c(O)c1)OC1CC(O)(C(=O)O)CC(O)C1O ; O=C(/C=C/c1ccc(O)c(O)c1)OC1C(O)CC(O)(C(=O)O)CC1O ; O=C(/C=C/c1ccc(O)c(O)c1)OC1CC(O)(C(=O)O)CC(O)C1O ; COc1cc(/C=C/C(=O)OC2CC(O)(C(=O)O)CC(O)C2O)ccc1O ; COc1cc(/C=C/C(=O)OC2C(O)CC(O)(C(=O)O)CC2O)ccc1O]

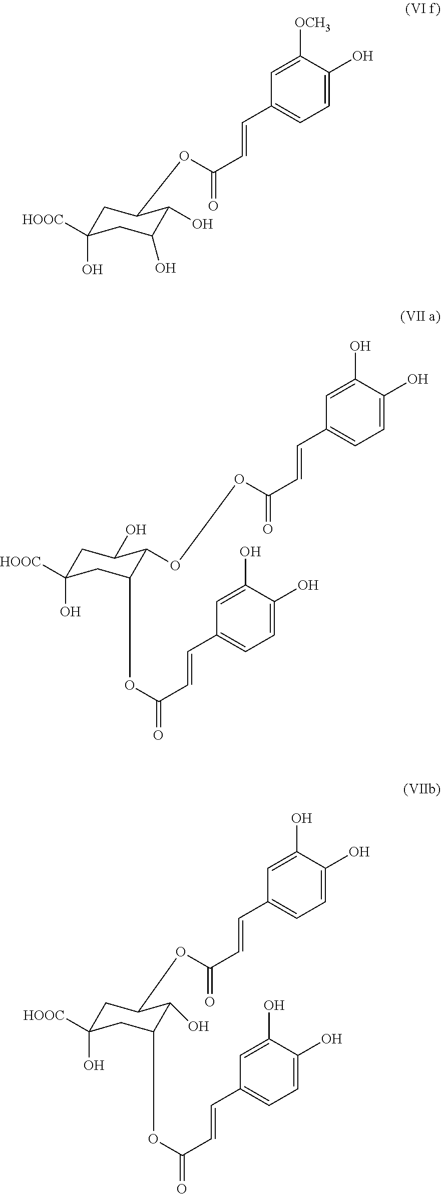 [SMILES: COc1cc(/C=C/C(=O)OC2CC(O)(C(=O)O)CC(O)C2O)ccc1O ; O=C(/C=C/c1ccc(O)c(O)c1)OOC1C(O)CC(O)(C(=O)O)CC1OC(=O)/C=C/c1ccc(O)c(O)c1 ; O=C(/C=C/c1ccc(O)c(O)c1)OC1CC(O)(C(=O)O)CC(OC(=O)/C=C/c2ccc(O)c(O)c2)C1O]

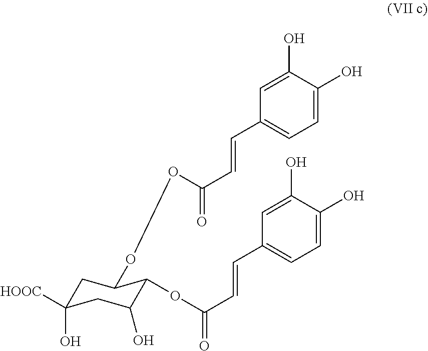 [SMILES: O=C(/C=C/c1ccc(O)c(O)c1)OOC1CC(O)(C(=O)O)CC(O)C1OC(=O)/C=C/c1ccc(O)c(O)c1]

In one embodiment, the molar ratio between the compound or the compounds having at least one 1,2,4-trioxane moiety and the chlorogenic acid or chlorogenic acids present in the combination according to the invention is for example between 2 and 0.002, preferably between 0.8 and 0.005, more preferably between 0.5 and 0.01 and even more preferably 0.1 and 0.01.

Chlorogenic acids may be used in their isolated form or as component of whole plants, plant parts or extracts of the aformentioned. As for Artemisia annua chlorogenic acids can be separated in a manner known per se to obtain the individual compounds for example, by partitioning between polyphasic solvent mixtures, recrystallization and/or chromatographic separation, for example over silica gel or by, e.g., medium pressure liquid chromatography over a reversed phase column or by fractional crystallization.

The individual chlorogenic acids can be worked up and/or purified according to standard methods, e.g., using chromatographic methods, distribution methods, (re-) crystallization, and the like.

Due to its high content of chlorogenic acid coffee, in particular roasted coffee can be used as a valuable source of chlorogenic acids.

In one embodiment the combinations according to the invention can be obtained by co-extracting coffee, in particular roasted coffee, with Artemisia annua, in particular dried leaves of Artemisia annua, e.g. with water, preferably at a temperature of 40 to 100° C., more preferably 50 to 100° C. The weight ratio of roasted coffee to Artemisia annua is for example from 1 to 50, preferably from 5 to 50. Coffees include those of the variety robusta (Coffea canephora) and arabica (Coffea arabica).

Suitable extracts also encompass teas comprising Artemisia annua, such as black teas, green teas and teas comprising licorice and cinnamon. Furthermore, the compounds of the combinations of the present invention, including their salts, can also be obtained in the form of their hydrates, or include other solvents used for their crystallization and/or extraction. The compounds of the combinations of the present invention may inherently or by design form solvates with pharmaceutically acceptable solvents including water; therefore, it is intended that the invention embrace both solvated and unsolvated forms. The term “solvate” refers to a molecular complex of a compound of the present invention including pharmaceutically acceptable salts thereof with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds having at least one 1,2,4-trioxane moiety comprised in the anti-cancer agents, or pharmaceutical compositions of the present invention, including salts, hydrates, and solvates thereof, may inherently or by design form polymorphs. All such polymorphs are encompassed by this invention.

As used herein, the terms “salt” or “salts” refers to an acid addition or base addition salt of a compound of the present invention. “Salts” include in particular “pharmaceutically acceptable salts”. The term “pharmaceutically acceptable salts” refers to salts that retain the biological effectiveness and properties of the compounds having at least one 1,2,4-trioxane moiety comprised in the anti-cancer agents or pharmaceutical compositions of this invention and, which typically are not biologically or otherwise undesirable. In some cases, the compounds having at least one 1,2,4-trioxane moiety comprised in the anti-cancer agents or pharmaceutical compositions of the present invention are capable of forming acid and/or base salts by virtue of the presence of amino and/or carboxyl groups like for artesunate or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, palmoate, phosphate/hydrogen phosphate/dihydrogen phosphate, polygalacturonate, propionate, stearate, succinate, sulfosalicylate, tartrate, tosylate and trifluoroacetate salts.

Inorganic bases from which salts can be derived include, for example, ammonium salts and metal cations from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable.

Lists of additional suitable salts can be found, e.g., in “Handbook of Pharmaceutical Salts: Properties, Selection, and Use” by Stahl and Wermuth (Wiley-VCH, Weinheim, Germany, 2002).

Any formula given herein is intended to represent unlabeled forms as well as isotopically labeled forms of the compounds of the combinations of the present invention having up to three atoms with non-natural isotope distributions, e.g., sites that are enriched in deuterium or 13C or 15N.

The compounds having at least one 1,2,4-trioxane moiety forming part of the anti-cancer agents or pharmaceutical compositions of the present invention may contain groups capable of acting as donors and/or acceptors for hydrogen bonds may be capable of forming co-crystals with suitable co-crystal formers. These co-crystals may be prepared from compounds of the present invention by known co-crystal forming procedures. Such procedures include grinding, heating, co-subliming, co-melting, or contacting in solution compounds of the present invention with the co-crystal former under crystallization conditions and isolating co-crystals thereby formed. Suitable co-crystal formers include those described in WO 2004/078163. Hence the invention further provides co-crystals comprising a compound comprising at least one 1,2,4-trioxane moiety.

The invention further comprises a pharmaceutical composition, comprising an anti-cancer agent of any of the preceding embodiments.

The invention also provides a method of preventing or treating cancer in a subject having cancer comprising administering therapeutically effective amounts of the anti-cancer agents or the pharmaceutical compositions according to the invention to a subject having cancer.

The invention also provides a method for prolonging survival of a subject having cancer comprising administering therapeutically effective amounts of the anti-cancer agents or pharmaceutical compositions according to the invention to a subject having cancer.

In a further embodiment, the invention provides a method of treating cancer in a subject comprising concurrently administering therapeutically effective amounts of the anti-cancer agents or of the pharmaceutical compositions according to the invention and at least one additional therapeutic agent to a subject having cancer. The inventive anti-cancer agents or the pharmaceutical composition and the additional therapeutic agent may be administered together or separately, with partially overlapping or fully overlapping periods of administration. Additional therapeutic agents may be selected from all agents known for this purpose and include e.g. anti-neoplastic agents and may be combined with the combinations or pharmaceutical compositions of this invention to create a single pharmaceutical dosage form. Alternatively these additional agents may be separately administered to the patient as part of a multiple dosage form, for example, using a kit.

In yet a further embodiment, the invention provides a method of treating cancer in a subject comprising sequentially administering therapeutically effective amounts of the the anti-cancer agents, in particular the combination according to the invention or of the pharmaceutical composition according to the invention and at least one additional therapeutic agent to a subject having cancer. The inventive anti-cancer agent, or pharmaceutical composition and the additional therapeutic agent may be administered with partially overlapping or non-overlapping periods of administration. Additional therapeutic agents may be selected from all agents known for this purpose and include e.g. anti-neoplastic agents such as Gemcitabine cisplatin, carboplatin and paclitaxel. It is an important finding of the invention as further outlined in the experimental part, that compounds comprising at least one 1,2,4-trioxane moiety, in particular artesunate and pharmaceutically acceptable salts thereof and thus the anti-cancer agents or pharmaceutical compositions according to the invention significantly improve the efficiency of known standard treatments for cancer in particular for ovarian cancer and lung cancer.

Therefore, in a further embodiment, the invention provides a method of for the treatment or of prolonging the survival of a subject having cancer comprising concurrently administering therapeutically effective amounts of the anti-cancer agent or the pharamaceutical composition according to the invention and at least one additional therapeutic agent to a subject having cancer. The inventive anti-cancer agent or pharmaceutical composition and the additional therapeutic agents may be administered together or separately, with partially overlapping or fully overlapping periods of administration. Additional therapeutic agents may be selected from all agents known for this purpose and include e.g. anti-neoplastic agents such as such as Gemcitabine, cisplatin, carboplatin and paclitaxel.

In another embodiment, the invention provides a method for the treatment or of prolonging the survival of a subject having cancer comprising sequentially administering therapeutically effective amounts of the anti-cancer agents or pharmaceutical compositions according to the invention and at least one additional therapeutic agent to a subject having cancer. The inventive anti-cancer agents or pharmaceutical compositions and the additional therapeutic agents may be administered with partially overlapping or non-overlapping periods of administration. Additional therapeutic agents may be selected from all agents known for this purpose and include e.g. anti-neoplastic agents such as Gemcitabine cisplatin, carboplatin and paclitaxel. In a specific embodiment the invention further comprises a method for the treatment or of prolonging the survival of a subject having cancer comprising administering to a subject in need thereof at least
(a) an anti-cancer agent or pharmaceutical composition according to the invention and
(b) an additional therapeutic agent selected from the group consisting of: a platinum-based doublet chemotherapy (PT-DC), wherein the PT-DC is a combination of (i) gemcitabine and cisplatin, such as Gemcitabine at a dose of 1250 mg/m² and cisplatin at a dose of at a dose of 75 mg/m², (ii) pemetrexed and cisplatin, such as pemetrexed at a dose of 500 mg/m² and cisplatin at a dose of 75 mg/m², or (iii) paclitaxel and carboplatin, such as paclitaxel at a dose of 200 mg/m² and carboplatin at a target area under the curve of dose of 6 mg/ml/min dose (AUC 6).

In a preferred embodiment the additional anti-cancer agent is a combination of paclitaxel and carboplatin, such as paclitaxel at a dose of 200 mg/m² and carboplatin at a target area under the curve of dose of 6 mg/ml/min dose (AUC 6).

The PT-DC is typically administered in 3-week cycles for up to a maximum of 6 cycles of chemotherapy. Chemotherapy treatment continues until disease progression, unacceptable toxicity or completion of the 4-6 cycles, whichever comes first.

Where the cancer is a non small cell lung cancer (NSCLC) the platinum-doublet chemotherapy regimens are typically dependent on NSCLC histology. Subjects with mixed histology are classified according to the predominant histology.

Squamous histology subjects may receive Gemcitabine (1250 mg/m²) with cisplatin (75 mg/m²); or Gemcitabine (1000 mg/m²) with carboplatin (AUC 5). Gemcitabine is administered on Day 1 and Day 8 of each cycle.

Non-squamous histology subjects may receive Pemetrexed (500 mg/m²) with cisplatin (75 mg/m²), administered on Day 1 of each cycle; or Pemetrexed (500 mg/m²) with carboplatin (AUC 6), administered on Day 1 of each cycle.

In a preferred embodiment the anti-cancer agents or pharmaceutical composition according to the invention are co-administered at the same day with the PT-DC.

Therefore, the invention further encompasses a kit for use in treating a subject having cancer, in paticular ovarian cancer or lung cancer, the kit comprising:
(a) a dosage, preferably a dosage as disclosed above of an anticancer-agent or pharmaceutical composition according tot he invention and
(b) a dosage of an additional therapeutic agent being a platinum-based doublet chemotherapy (PT-DC) and
(c) instructions for using the anti-cancer agent or pharmaceutical composition accorsing to the invention and the other therapeutic agent.

It is a further finding of the invention, as evidenced by the examples below, that the anti-cancer agents, pharmaceutical compositions, in particular artesunate and pharmaceutically acceptable salts thereof are even synergistically effective in the treatment of NSCLC with mutations of KEAP1 or NFE2L2 (the gene encoding NRF2) when applied together with an NRF2 inhibitor such as ML 385 (N-[4-[2,3-Dihydro-1-(2-methylbenzoyl)-1H-indol-5-yl]-5-methyl-2-thiazolyl]-1,3-benzodioxole-5-acetamide).

In a specific embodiment the invention therefore further comprises a method for the treatment or of prolonging the survival of a subject having cancer, in particular of NSCLC, preferably those with mutations of KEAP1 or NFE2L2, comprising administering to a subject in need thereof at least
(a) an anti-cancer agent or pharmaceutical composition according to the invention and
(b) an additional therapeutic agent selected from the group consisting of NRF2 inhibitors such as ML385.

The invention further encompasses an enhanced pharmaceutical composition or a kit for use in treating a subject having cancer, in paticular non-small cell lung cancer, the kit comprising:
(a) a dosage, preferably a dosage as disclosed above of an anticancer-agent or pharmaceutical composition according to the invention and
(b) a dosage of a NRF2 inhibitor and additionally for the kits
(c) instructions for using the anti-cancer agent or pharmaceutical composition according to the invention and the NRF2 inhibitor.

An additional aspect of this invention encompasses an article of manufacture comprising an anti-cancer agent or pharmaceutical compositions or kits as described hereinabove to treat or prevent cancer or to prolong the survival of a subject having cancer; and packaging material comprising a label which indicates that the composition can be used to treat or prevent cancer or to prolong the survival of a subject having cancer.

This invention further encompasses the anti-cancer agents and pharmaceutical compositions and kits as described herein for use as a medicament, in particular for treating or preventing cancer or for the prolongation of survical of a subject having cancer.

Yet another aspect of this invention relates to a method of destructing or inhibiting the growth of cancer cells comprising exposing the cancer cells to an effective amount of the anti-cancer agents or pharmaceutical compositions described herein either with or without additional therapeutic agents. This method can be practiced in vitro or in vivo.

The scope of the invention further includes the use of the anti-cancer agents or pharmaceutical compositions to prevent or inhibit the growth of cancer cells, cell division of cancer cells or metastasis of cancer either in combination with additional therapeutic agents or not.

The dose range of the anti-cancer agentsd or pharmaceutical compositions of the invention applicable per day is for example from 0.1 to 100 mg/kg of body weight, preferably from 0.1 to 50 mg/kg of body weight, and even more preferably from 0.5 to 50 mg/kg of body weight calculated on the sum of compounds having at least one 1,2,4-trioxane moiety of the anti-cancer agents or pharmaceutical compositions described and defined herein.

The same applies to the kits as defined hereinabove.

Each dosage unit may contain from 5% to 95 wt-% of the anti-cancer agents or pharmaceutical compositions of the invention. Preferably the pharmaceutical compositions according to the invention contain from 20% to 80 wt-% of the anti-cancer agents according to the invention.

In another embodiment the pharmaceutical compositions according to the invention contain from 20% to 80 wt-% of compounds having at least one 1,2,4-trioxane moiety.

The actual pharmaceutically effective amount or therapeutic dosage will of course depend on factors known by those skilled in the art such as age and weight of the patient, route of administration, and severity of disease. In any case the combination will be administered at dosages and in a manner which allows a pharmaceutically effective amount to be delivered based upon patient's individual condition.

When the pharmaceutical composition of this invention comprises the inventive anti-cancer agents and one or more additional therapeutic agents, both the anti-cancer agents and the additional agent should be present at dosage levels of between about 10 to 100%, and more preferably between about 10 and 80% of the dosage normally administered in a monotherapy regimen.

The compounds of formulae (VIII) to (XVIII) below were reported to be present in Artemisia annua extracts, see inter alia Czechowski et al., Frontiers in Plant Science, 2019, Vol. 10, Article 984; Zarelli et al., Phytochemical Analysis 2019, 30, 564-571.

Therefore, the anti-cancer agents, pharmaceutical compositions and kits according to the invention may further include at least one compound, for example one, two, three, four, five, six, seven, eight, nine, ten or all compounds selected from the group consisting of those of formulae (VIII) to (XVIII)

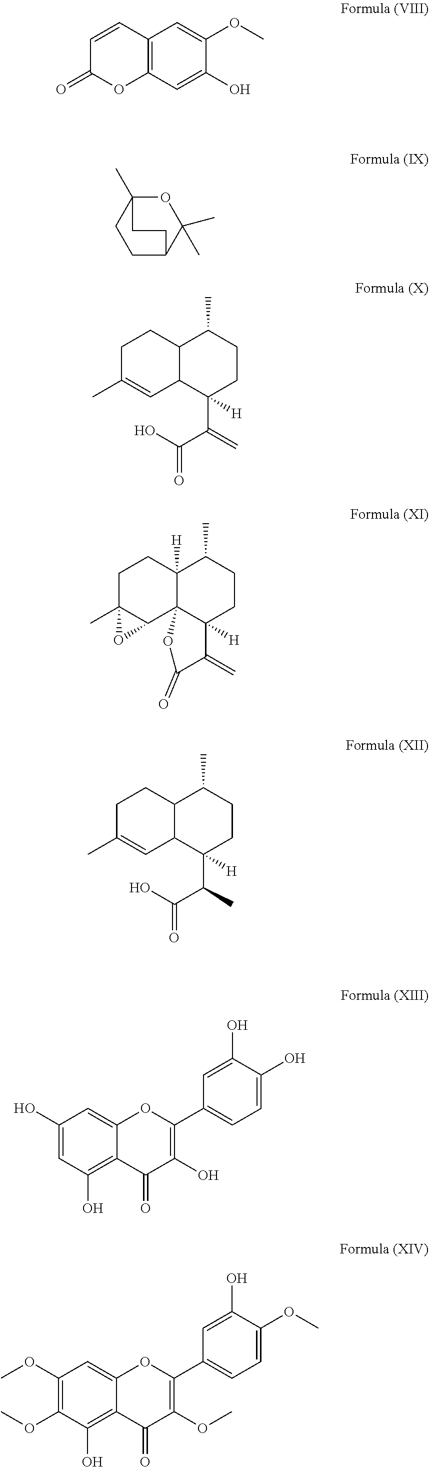

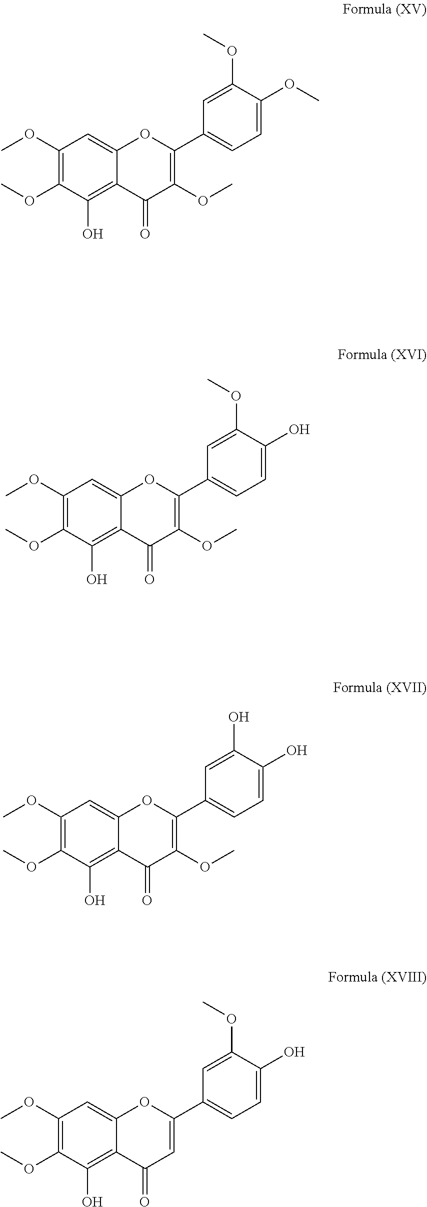

The compound of formula (VIII) is scopoletin.

The compound of formula (IX) is 1,8-cineole.

The compound of formula (X) is artemisinic acid.

The compound of formula (XI) is arteannuin-B.

The compound of formula (XII) is dihydroartemisinic acid.

The compound of formula (XIII) is fisetin.

The compound of formula (XIV) is casticin.

The compound of formula (XIV) is artemetin.

The compound of formula (XVI) is chrysoplenetin.

The compound of formula (XVII) is chrysoplenol-D.

The compound of formula (XVIII) is cirsilineol.

The pharmaceutical compositions of the invention can be administered by known methods, including oral, parenteral, inhalation, and the like. In certain embodiments, the compound of the invention is administered orally, as a pill, lozenge, troche, capsule, solution or extract such as a tea or other type of infusion beverage, in particular extracts of Artemisia annua, or suspension.

In other embodiments, pharmaceutical compositions of the invention are administered by injection or infusion. Infusion is typically performed intravenously, often over a period of time between about 15 minutes and 4 hours. In other embodiments, pharmaceutical compositions of the invention are administered intranasally or by inhalation; inhalation methods are particularly useful. Pharmaceutical compositions of the invention the present invention exhibit oral bioavailability, so oral administration is sometimes preferred.

An “effective amount” of a compound is that amount necessary or sufficient to treat or prevent cancer or to prolong survival of a subject having cancer.

The effective amount can vary depending on such factors as the size and weight of the subject, the type and severity of illness, or the particular compound of the invention. For example, the choice of the combination or pharmaceutical composition according to the invention can affect what constitutes an “effective amount.” One of ordinary skill in the art would be able to study the factors contained herein and make the determination regarding the effective amount of the combination or pharmaceutical composition of the invention without undue experimentation.

The regimen of administration can affect what constitutes an effective amount. The combination or pharmaceutical composition of the invention can be administered to the subject either prior to or after the onset of cancer. Further, several divided dosages, as well as staggered dosages, can be administered daily or sequentially, or the dose can be continuously infused, or can be a bolus injection. Further, the dosages of the combination or pharmaceutical composition of the invention can be proportionally increased or decreased as indicated by the exigencies of the therapeutic or prophylactic situation.

The combinations of the invention may be used in the treatment of states, disorders, or diseases as described herein, or for the manufacture of pharmaceutical compositions for use in the treatment of cancer. The invention provides methods of use of combinations of the present invention in the treatment of these diseases or for preparation of pharmaceutical compositions comprising such combinations of the present invention for the treatment of these diseases.

The language “pharmaceutical composition” includes preparations suitable for administration to mammals, e.g., humans. When the combinations of the present invention are administered as pharmaceuticals to mammals, e.g., humans, they can be given per se or as a pharmaceutical composition containing, for example, 0.1 to 99.5% (more preferably, 0.5 to 90%) of the combination of the invention or any subgenus thereof as active ingredient in combination with a pharmaceutically acceptable carrier, or optionally two or more pharmaceutically acceptable carriers.

The phrase “pharmaceutically acceptable carrier” is art recognized and includes a pharmaceutically acceptable material, composition or vehicle, suitable for administering the compounds of the inventive combinations to mammals. The carriers include liquid or solid filler, diluent, excipient, solvent, or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Typically, pharmaceutically acceptable carriers are sterilized and/or substantially pyrogen-free.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, inhalation, topical, transdermal, buccal, sublingual, rectal, vaginal, and/or parenteral administration. The formulations may conveniently be presented in unit dosage form and may be prepared by any methods well known in the art of pharmacy. The amount of active ingredient that can be combined with a carrier material to produce a single dosage form will generally be that amount of the combination or pharmaceutical composition that produces a therapeutic effect. Generally, out of one hundred percent, this amount will range from about 1 percent to about ninety-nine percent of active ingredient, preferably from about 5 percent to about 80 percent.

Methods of preparing these formulations or compositions include the step of bringing into association a compound of the present invention with the carrier and, optionally, one or more accessory ingredients. In general, the formulations are prepared by uniformly and intimately bringing into association a combination or pharmaceutical composition of the present invention with liquid carriers, or finely divided solid carriers, or both, and then, if necessary, shaping the product.

Formulations of the invention suitable for oral administration may be in the form of capsules, cachets, pills, tablets, lozenges (using a flavored base, for example, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary, or paste.

In solid dosage forms of the invention for oral administration such as capsules, tablets, pills, dragees, powders, granules and the like, the combinations or pharmaceutical compositions are mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, teas, coffees, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

Pharmaceutical compositions of this invention suitable for parenteral administration may comprise one or more compounds of the invention in combination with one or more pharmaceutically acceptable carriers such as sterile isotonic aqueous or nonaqueous such as ethanolic solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

The preparations of the present invention may be given orally, pulonary, parenterally, topically, or rectally.

They are of course given by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Intravenous infusion is sometimes a preferred method of delivery for compounds of the invention. Infusion may be used to deliver a single daily dose or multiple doses. In some embodiments, the anti-cancer agents or pharmaceutical compositions of the invention are administered by infusion over an interval between 15 minutes and 4 hours, typically between 0.5 and 3 hours. Such infusion may be used once per day, twice per day, or up to three times per day.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The anti-cancer agents or pharmaceutical compositions may be administered to humans and other animals for therapy by any suitable route of administration, including orally, pulmonary, nasally, as by, for example, a spray, rectally, intravaginally, parenterally, intracisternally and topically, as by powders, ointments or drops, including buccally and sublingually.

Regardless of the route of administration selected, the anti-cancer agents or pharmaceutical compositions of the present invention, which may be used in a suitable hydrated form, and/or the pharmaceutical compositions or kits of the present invention, are formulated into pharmaceutically acceptable dosage forms by conventional methods known to those of skill in the art.

Actual dosage levels of the active ingredients in the pharmaceutical compositions and kits of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition or kit required. For example, the physician or veterinarian could start doses of the anti-cancer agents of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above.

If desired, the effective daily dose of the active compound may be administered as a single dose per day, or as two, three, four, five, six, or more sub-doses administered separately at appropriate intervals throughout the day, optionally, in unit dosage forms. Compounds delivered orally or by inhalation, are commonly administered in one to four doses per day. Compounds delivered by injection are typically administered once per day, or once every other day. Compounds delivered by infusion are typically administered in one to three doses per day. When multiple doses are administered within a day, the doses may be administered at intervals of about 4 hours, about 6 hours, about 8 hours, or about 12 hours.

While it is possible for a anti-cancer agent of the present invention to be administered alone, it is preferable to administer it as a pharmaceutical composition such as those described herein. Thus, methods of using the anti-cancer agents of the invention include administering the anti-cancer agents as a pharmaceutical composition, wherein at least one compound having at least one 1,2,4-trioxane moiety is admixed with a pharmaceutically acceptable carrier prior to administration.

The invention further encompasses the use of the anti-cancer agents, pharmaceutical compositions or kits of the invention in combination with immunomodulators.

The combinations and pharmaceutical compositions described herein can be used or administered in combination with one or more therapeutic agents that act as immunomodulators, e.g., an activator of a co-stimulatory molecule, or an inhibitor of an immune-inhibitory molecule, or a vaccine.

By “in combination with,” it is not intended to imply that the therapy or the therapeutic agents must be administered at the same time and/or formulated for delivery together, although these methods of delivery are within the scope described herein. The immunomodulator can be administered concurrently with, prior to, or subsequent to, one or more compounds of the invention, and optionally one or more additional therapies or therapeutic agents. The therapeutic agents in the combination can be administered in any order. In general, each agent will be administered at a dose and/or on a time schedule determined for that agent. It will further be appreciated that the therapeutic agents utilized in this combination may be administered together in a single composition or administered separately in different compositions. In general, it is expected that each of the therapeutic agents utilized in combination be utilized at levels that do not exceed the levels at which they are utilized individually.

The present invention is further illustrated by the following examples which should not be construed as further limiting. The contents of all references cited throughout this application are expressly incorporated herein by reference.

### Examples

A) Ovarian Cancer Cell Lines

Materials and Methods

1.1 Cell Culture and Artesunate

Commercially available, human ovarian cancer cell lines UWB1.289 (ATCC CRL-2945), Caov-3 (ATCC HTB-75), and OVCAR-3 (ATCC HTB-161) were obtained from ATCC. Cell lines were cultured and maintained in, cell line-specific, complete growth media as recommended by ATCC. All cells were incubated at 37° C. in 5% CO₂. Artesunate was purchased from MedChem Express and dissolved in DMSO as a 200 mM stock solution and stored at −80° C. Artesunate was serially diluted in DMSO, and then media, to the desired concentrations at the time of each experiment.

1.2 Cell Viability Assays

White-walled 96-well microplates were seeded at 3×10³ cells per well in 100 μL growth media and incubated for 24 h at 37° C., 5% CO₂. The growth media was removed and replaced with fresh media containing serially diluted drugs or drugs of interest. Each drug concentration was tested in duplicate and vehicle (0.1% DMSO) media for control, in triplicate assays. Twelve dilutions of the artesunate stock solution, ranging from 0.0011-200 μM were used to treat cells and incubated them for 72 h. Cell viability was assessed using a CellTiter-Glo 2.0 viability assay (Promega) and luminescence was measured using a Varioskan LUX multimode microplate reader (ThermoFisher Scientific). Percent viability was calculated by normalizing the relative luminescence signal of each treated well to the matched vehicle controls. After graphing the calculated percent viability for each artesunate concentration, a four-parameter log-logistic model was used to fit a non-linear regression line and the IC50 was calculated for each cell line using GraphPad Prism 5.01.

1.3 DNA Damage Assay

Caov-3 cells were seeded into black-walled μClear 96-well plates at a density of 4000 cells per well in 100 μL of complete growth media and allowed to adhere for 24 hrs at 37° C. with 5% CO₂. The media was removed and replaced with complete media containing 5 μM, 10 μM, 50 μM, 100 μM artesunate, 0.1% DMSO as a negative control, or 25 μM cisplatin as a positive control. Cells were incubated with drugs for 48 hrs and then fixed for 15 min at room temperature in 4% paraformaldehyde. 0.25% Triton X-100 was used to permeabilize the cells for 15 min and then blocked in 0.1% bovine serum albumin (BSA) for one hour. DNA damage was assessed with immunofluorescent staining for phosphorylated histone H2AX (pH2AX) using the HCS DNA Damage Kit (Invitrogen). The Cell-Insight CX7 High Content Analysis Platform was used for imaging and HCS Studio software to quantify the nuclear pH2AX signal (both ThermoScientific). The statistical analysis of pH2AX signal was completed on GraphPad Prism (version 5.01).

1.4 Flow Cytometric Analysis of Cell Cycle

UWB1 and Caov-3 cells were propagsted in culture flasks under standard cell culture conditions outlined above. For the staining solution, 2 mg DNase-free Rnase A (Sigma) and 200 μL of 1 mg/mL propidium iodide were added to 10 mL of 0.1% (v/v) Triton X-100 in PBS. Treatment media was made by serially diluting stock 200 μM artesunate solutions into cell line-specific media on the day of treatment. Once cell cultures reached confluence, we removed the growth media from the treatment flask and added the artesunate treatment media at a concentration of M. Cells were collected after 24 or 48 h, washed in PBS, and resuspended in 0.5 mL PBS before transferring to tubes containing 4.5 mL 70% EtOH for fixation. Cells were fixed at least overnight at −20° C. Fixed cells were washed in PBS and resuspended in 1 mL of staining solution. PI stained cells were sorted with the LSR II cell analyzer. The analysis was performed with ModFit LT v3.3 software. Statistical analysis comparing the percentage of cells in each phase of the cell cycle (G1, S, or G2) was performed using GraphPad Prism (version 5.01).

1.5 Drug Administration Sequence Assay

In a similar fashion to the protocols mentioned above for Cell Viability Assays, cells were plated in a standard 96-well plate at a cell density of 3000 cells/100 μL and incubated for 24 h. Artesunate, carboplatin, and paclitaxel stock solutions were diluted with DMSO and media to achieve a final concentration of 40 μM, 16 μM, and 32 μM, respectively. Each drug was added, as indicated, for 24 h and treatment media were then replaced with fresh media. Drug administration sequences were artesunate on day 1 (D1A) or day 2 (D2A), carboplatin and paclitaxel on day 2 (D2C/T), carboplatin, paclitaxel, and artesunate on day 2 (D2C/T/A), or artesunate on day 1 followed by carboplatin and paclitaxel on day 2 (D1A; D2C/T). Cells were incubated at standard growth conditions for a total of 72 h. Viability measurements were determined using the CellTiter-Glo 2.0 viability assay (Promega). Luminescence was measured using a Varioskan LUX multimode microplate reader (ThermoFisher Scientific). Statistical analysis was performed using GraphPad Prism (v5.01).

Results

1.6 Determination of Antineoplastic Activity

To evaluate the antineoplastic activity of artesunate in ovarian cancer, the dose-dependent effect of artesunate on the viability of three epithelial ovarian cancer cell lines was determined: Caov-3 (adenocarcinoma), UWB1.289 (high-grade serous carcinoma with a BRCA1 mutation), and OVCAR-3 (adenocarcinoma) using the CellTiter-Glo 2.0 assay. The IC50 of artesunate in all three cell lines was in the low to mid micromolar range; specifically, the IC50 was 26.91 μM (95% confidence interval 6.287-115.2 μM) in UWB1, 15.17 μM (10.49-21.93 μM) in Caov-3, and 4.67 μM (3.280-6.638 μM) in OVCAR-3 cells. A One-way ANOVA analysis showed no significant difference (p>0.05) in the IC50 between these three cell lines. The IC50 detected for all three ovarian cancer cell lines tested is consistent with previously established findings (see [6] as cited above) and within range of therapeutically achievable in vivo plasma concentrations (approximately 20 μM) (see [14] as cited above).

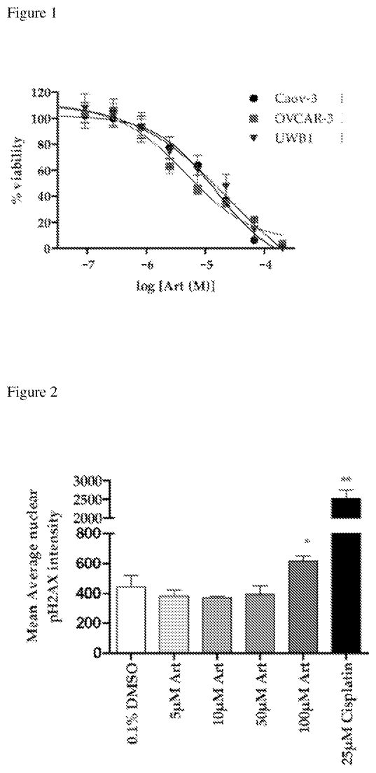

The detailed results are shown in FIG. 1.

1.7 Assessment of the Primary Mode of Action

Caov-3 cells were chosen due to their close correlation to IC50s previously published as well as their known platinum sensitivity which correlates to most high grade serous ovarian cancers. To assess the ability of artesunate to induce DNA damage in Caov-3 cells, the immunofluorescent staining for pH2AX was quantified, a marker of double-strand breaks, following a 48 hr treatment with concentrations of artesunate ranging from 5-100 μM and 25 μM cisplatin as a positive control.

The Results are Shown in FIG. 2.

Cells treated with 0.1% DMSO as a control had a mean pH2AX signal of 443.2+/−76.38, while treatment with 25 μM cisplatin resulted in a mean signal of 2517+/−230.4. The only artesunate treatment that significantly increased DNA damage was 100 μM artesunate with a mean pH2AX signal of 617.0+/−31.96. Treatment with 5, 10, or 50 μM resulted in pH2AX measurements of 382.8+/−39.58, 370.2+/−9.283, and 393.8+/−58.79, respectively. Only the highest concentration of artesunate (100 μM) and cisplatin resulted in a significant increase in DNA damage compared to vehicle control-treated cells as assessed by a one-tailed unpaired t-test p=0.0486 (100 μM artesunate) and p=0.0034 (25 μM cisplatin).

Therefore, while artesunate was able to induce DNA damage, this effect was hardly ovserved at clinically relevant concentrations in Caov-3 cells.

The second mechanism of action for artesunate investigated was the induction of cell cycle arrest. The cell cycle progression by propidium iodide staining and flow cytometry of Caov-3 and UWB1 cells following treatment with 10 μM artesunate or 0.1% DMSO (vehicle control) for 24 and 48 h was assessed. The percentage of cells in G1 and S phase with and without 10 μM artesunate treatment was determined from three independent experiments. In Caov-3 cells, 48-h artesunate treatment resulted in an increased percentage of cells in G1, 78.98%+/−0.9546 compared to 61.23%+/−1.789, in vehicle-treated cells. UWB1 cells had 65.35%+/−0.0849 cells in G1 after artesunate treatment compared to 60.35%+/−2.418 of control cells

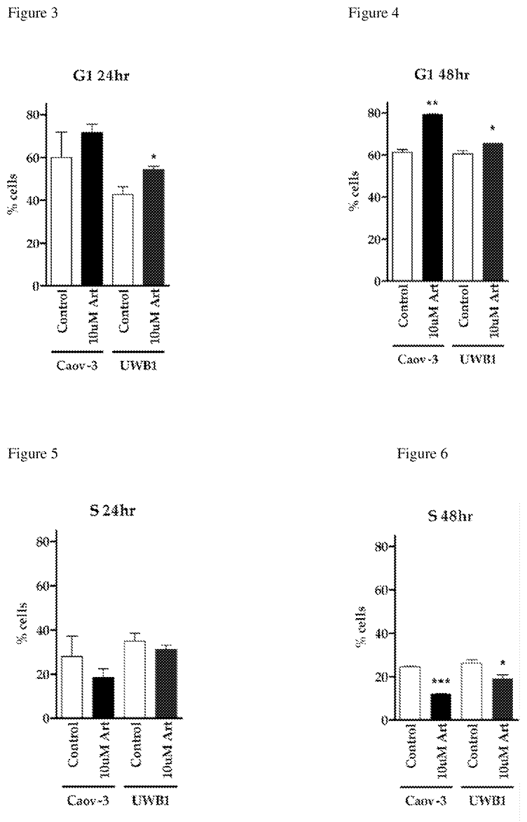

The results are shown in FIGS. 3 and 4.

Both cell lines showed a significant increase in cells in G1 with p=0.0032 in Caov-3 and p=0.0499 in UWB1 (one-tailed unpaired t-test). In further subgroup analysis, the increased percentage of Caov-3 and UWB1 cells in the G1 phase following treatment with artesunate was accompanied by a significant decrease in cells in the S phase, p=0.0009 and p=0.0497, respectively. In Caov-3 cells, 48-h artesunate treatment resulted in a decreased percentage of cells in S phase, 12.14%+/−0.1556 compared to 24.42%+/−0.7354 in vehicle-treated cells. UWB1 cells had 18.87%+/−2.779 cells in S phase after artesunate treatment compared to 26.25%+/−2.234 of control cells

The results are shown in FIGS. 5 and 6.

These experiments demonstrate that, in the cell lines tested, clinically relevant concentrations of artesunate induce G1 arrest.

1.8 Addition of Artesunate to Carboplatin and Paclitaxel

The current primary treatment regimen in ovarian cancer is a platinum/taxane doublet, such as carboplatin and paclitaxel. In view of the favorable side effect profile and its effects on cell viability make the effect of adding artesunate to cells treated with both paclitaxel and carboplatin was assessed. Since artesunate induces cell cycle arrest in the G1-phase, artesunate was evaluated as either a pretreatment (24 h prior) or concurrently with carboplatin and paclitaxel. In both cell lines, treatment with artesunate alone on either day 1 or day 2 resulted in a decreased cell viability to 54.60-63.43% compared to control-vehicle treated cells.

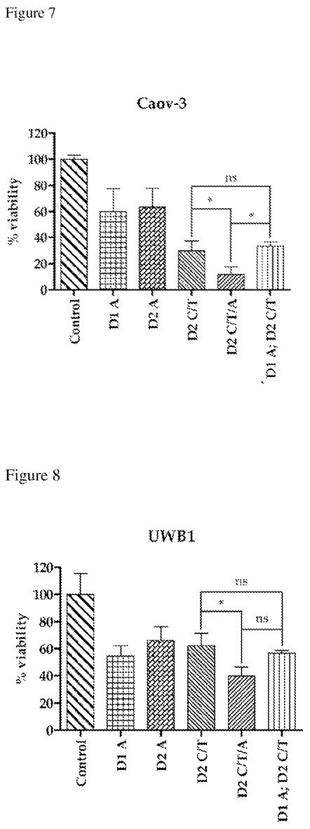

The results are shown in FIGS. 7 and 8.

In Caov-3 cells, treatment with carboplatin/paclitaxel resulted in reduced cell viability to 29.60%+/−7.780, which was significantly decreased when artesunate was added concurrently (11.55%+/−5.917, p<0.05 One-way ANOVA) but not when cells were pretreated with artesunate (33.16%+/−3.349, p>0.005).

Similarly, in UWB1, cell viability was decreased to 62.06%+/−9.389 when treated with carboplatin and paclitaxel that was further decreased to 39.65%+/−6.850 (p<0.05, One-way ANOVA) with the concurrent addition of 40 μM artesunate.

In these cells, pretreatment with artesunate resulted in cell viability of 56.41%+/−2.148, which was not statistically significant when compared to cells treated with carboplatin and paclitaxel.

It is a major finding that concurrent treatment of artesunate with carboplatin and paclitaxel showns a significant improvement of the standard regimens effectiveness.

B) Lung Cancer Cell Lines

Materials and Methods

2.1. Cell Lines and Reagents

A549 (ATCC: CCL-185), H1299 (ATCC: CRL-5803), and H1563 (ATCC: CRL-5875) NSCLC cell lines were purchased directly from ATCC. All cell lines were initially expanded and low passage numbers aliquots were frozen back to ensure experiments were conducted in cell lines with similar passage numbers. Cell lines were screened for mycoplasma at regular intervals, including when cell lines were frozen back. All cells were grown in RPMI 1640 (Lonza, Basel, Switzerland: 12-167F) with 10% Fetal Bovine Serum (Sigma-Aldrich, St. Louis Mo., USA: F0926), Penicillin/Streptomycin (Gibco, Waltham Mass., USA: 15140-122), and 2 mM Glutamax (Gibco: 35050-061) and were maintained in a 37° C. humidified incubator with 5% CO₂. Purified artesunate (HY-N0193) was purchased from MedChem Express (Monmouth Junction NJ, USA) and ML385 (SML1833) was purchased from Sigma-Aldrich.

2.2. Drug Response Assays

Cells are seeded into white-walled 96-well plates at 2500 cells (A549 and H1299) or 4000 cells (H1563) per well in 100 μL of complete growth media and allowed to adhere for 24 h at 37° C. with 5% CO₂. After 24 h, artesunate is serially diluted 1:3 in DMSO to obtain 12 drug stocks in 100% DMSO; subsequently, each stock is diluted 1:1000 in complete media so the final concentration of DMSO is 0.1%. Growth media is aspirated off the cells and replaced with media containing the diluted artesunate, with each drug concentration being tested on duplicate wells; additionally, triplicate wells receive media with only 0.1% DMSO and serve as untreated controls. Cells are incubated with drugs for 96 h prior to using CellTiter-Glo 2.0 (Promega, Madison Wis., USA: G9243) to assess cell viability. Data is presented as the percent viability of treated cells normalized to 0.1% DMSO-treated control cells. GraphPad Prism (version 5.01) was used to fit a dose response curve (four parameter log-logistic model) to the data and to calculate IC50 values. For studies assessing artesunate in combination with ML385, artesunate drug stocks were diluted in media containing 5 μM ML385 and the matched control wells were treated with 5 μM ML385.

2.3. DNA Damage Assay

A549 and H1299 cells are seeded into black-walled μClear 96-well plates (ThermoScientific, Waltham Mass., USA: 165305) at a density of 3000 cells per well in 100 μL of complete growth media and allowed to adhere for 24 h at 37° C. with 5% CO2. Subsequently, media was removed and replaced with complete media containing 5 μM, 10 μM, 50 μM, 100 μM Artesunate, 0.1% DMSO as a negative control or 25 μM Cisplatin (Tocris Bioscience, Minneapolis Minn., USA: 2251) as a positive control. Cells were incubated with drugs for 48 h and then fixed for 15 min at RT in 4% paraformaldehyde (Alfa Aesar, Tewksbury MA, USA: J61899). Following fixation, cells were permeabilized with 0.25% Triton X-100 (Alfa Aesar: A16046) for 15 min and blocked in 0.1% bovine serum albumin (BSA) for 1 hr. DNA damage was assessed by immunofluorescence staining for phosphorylated histone H2AX (pH2AX) using the HCS DNA Damage Kit (Invitrogen, Waltham Mass., USA: H10292). Cells were imaged using the CellInsight CX7 High Content Analysis Platform (ThermoScientific: CX7A1110) and quantification of nuclear pH2AX signal was performed using the HCS Studio software (ThermoScientific). Statistical analysis of pH2AX signal was performed on GraphPad Prism (version 5.01). The effect of the combination treatment of artesunate and ML385 on DNA damage was also assessed using the HCS DNA Damage Kit. The day after cells were seeded, as above, they were treated with DMSO or 5 μM ML385 for 24 h. The next day, the media was aspirated and fresh media containing 10 μM, 50 μM, 100 μM artesunate, 0.1% DMSO as a negative control or 25 μM Cisplatin with or without fresh 5 μM ML385 was added to the cells. After 24 h, the cells were fixed with 4% paraformaldehyde and DNA damage was assessed as described above.

2.4. Western Blotting

A549 and H1299 cells were treated with 10 μM artesunate for 0, 6, or 24 h prior to being lysed in RIPA buffer (Pierce, Waltham Mass., USA: 89900) containing Halt Protease and Phosphatase Inhibitor Cocktail (ThermoScientific: 78441) and Benzonase Nuclease (Sigma: E1014), incubated on ice for 10 min, and cleared by centrifugation. Protein concentrations were determined using a BCA Protein Assay (Pierce: 23227) and 40 μg total protein was loaded onto a NuPAGE 4-12% Bis-Tris Gel (Life Technologies, Waltham Mass., USA: NP0321BOX) for electrophoresis prior to transfer to a PVDF membrane (Invitrogen: LC2005) for blotting. Antibodies against KEAP1 (Cell Signaling, Danvers Mass., USA: 8047S) and NQO-1 (Cell Signaling: 3187S) were purchased from Cell Signaling, while antibodies to β-ACTIN were purchased from R&D Systems (MAB8929). IRDye-conjugated secondary antibodies were purchased from LI-COR (925-32213 and 925-68070) and Westerns blots were imaged using a LI-COR Odyssey imaging system.

2.5. siRNA Knockdown

Two wells of a 6-well plate were seeded with 2×10⁵ A549 or H1299 cells per well and allowed to adhere overnight before transfection with 5 nM siGENOME Control pool non-targeting #2 (Dharmacon, Boulder Colo., USA: D-001206-14-5) or siGENOME SMARTpool siRNA targeting human KEAP1 (GGACAAACCGCCUUAAUUC; CAGCAGAACUGUACCUGUU; GGGCGUGGCUGUCCUCAAU; CGAAUGAUCACAGCAAUGA) purchased from Dharmacon (M-012453-00-0005). After 24 h, cells were seeded into a 96-well plate at 2500 cells per well or were seeded into 60 mm tissue culture plates to assess knockdown efficiency by Western blot. Then 48 h after initial transfections, cells seeded into 96-well plates were treated as detailed above (Drug response assays) to assess the effect of KEAP1 knockdown on drug sensitivity, while the 60 mm plates were harvested in RIPA buffer for Western blotting as described above to determine knockdown efficiency. To determine whether knocking down KEAP1 altered artesunate's ability to induce DNA damage in NSCLC cells, cells were transfected with 5 nM non-targeting control or siRNA targeting human KEAP1. The next day, 6000 cells per well were seeded in complete growth media in a black-walled clear bottom 96-well plate and a DNA damage assay was performed as described above, 2.3. DNA Damage Assay.

2.6. Synergy or Artesunate and NRF2 Inhibitors

To assess drug interactions, drug response assays were preformed similarly to the method above; however, a 6×6 matrix design was used to assay pairs of drugs alone and in combination with five serially diluted concentrations of each drug. Cell viability was assessed following a 96 h treatment using CellTiter-Glo 2.0. Each well was normalized to untreated control cells which were grown in media with 0.2% DMSO and the percentage of viable cells was determined. R statistical software, specifically the synergyfinder package (version 1.10.4)), was used to generate a synergy score using the Bliss independence model and the Zero Interaction Potency (ZIP) model (see Bliss, C. I. The Toxicity of Poisons Applied Jointly1. Ann. Appl. Biol. 1939, 26, 585-615, doi:10.1111/j.1744-7348.1939.tb06990.x, Yadav, B.; Wennerberg, K.; Aittokallio, T.; Tang, J. Searching for Drug Synergy in Complex Dose-Response Landscapes Using an Interaction Potency Model. Comput. Struct. Biotechnol. J. 2015, 13, 504-513, doi:10.1016/j.csbj.2015.09.001.)

2.7. Statistical Analysis

To assess whether the difference in artesunate IC50 values were statistically significant, the IC50 values calculated from multiple independent experiments along with the standard error for each experiment were graphed using GraphPad Prism 5. A two-tailed t-test was used to determine whether the mean IC50 between two different cell lines or between treated and control cells were statistically different. P-values are reported in the figure legends and results with a p-value less than 0.05 indicating a statistical significant difference. Differences in DNA damage was determined for each cell line using a one-way ANOVA followed by Dunnett's multiple comparison test to compare each treatment to 0.1% DMSO control for cells treated with artesunate alone or in combination with 5 μM ML385. A two-way ANOVA with Bonferroni post test was utilized to assess DNA damage following 24 h artesunate treatment in cells transfected with siRNA.

Results

2.8 Artesunate Sensitivity

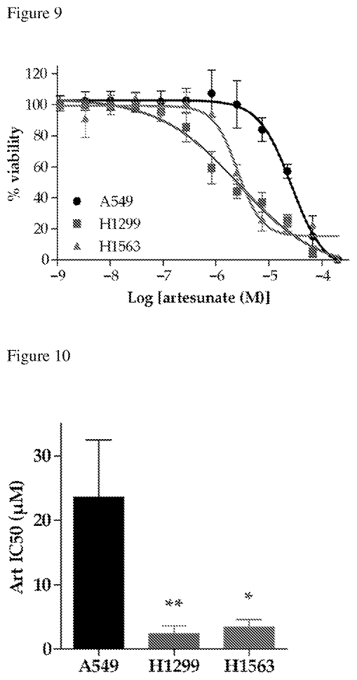

First, the effect of artesunate on three NSCLC cell lines (A549, H1299, and H1563) was investigated by assessing cell viability, using a CellTiter-Glo 2.0 assay, after treatment with increasing concentrations of artesunate for 96 h. The cell lines separated as either sensitive or resistant with a 10-fold difference in IC50 values for artesunate. A549 cells were less sensitive to artesunate, with a mean IC50 of 23.63 μM±8.886 μM from three independent experiments, while H1299 and H1563 cells were sensitive to artesunate, with mean IC50s of 2.36 μM±1.275 μM and 3.43 μM±1.190 μM, respectively, (see FIGS. 9 and 10).

2.9 Generation of Reactive Oxygen Species

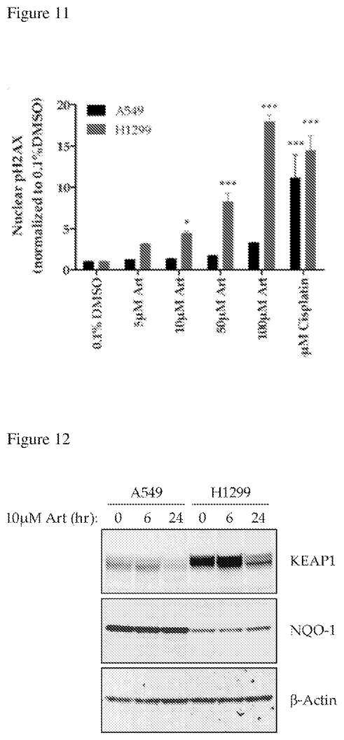

It is known that Artesunate's primary mechanism of action against malaria is to generate reactive oxygen species (ROS) through a reaction between the endoperoxide bridge within artesunate and heme iron in the malaria plasmodium. Within cancer cells, the generation of ROS can cause DNA damage through double-strand breaks which can be assessed by immunofluorescent staining for phosphorylated histone H2AX (pH2AX); therefore, the ability of artesunate to induce DNA damage in A549 (less sensitive) and H1299 (sensitive) NSCLC cell lines in a dose-dependent manor was assessed. Treatment with 25 μM cisplatin was used as a positive control for DNA damage while 0.1% DMSO was used as a negative/vehicle control. The mean nuclear pH2AX signal intensity for each treatment was normalized to the matched 0.1% DMSO control and plotted as the ratio of treatment/0.1% DMSO (dimethyl sulfoxide)±SD, (see FIG. 11). In both A549 and H1299 cells, cisplatin induced significantly more (p<0.001) pH2AX staining than what was observed in matched 0.1% DMSO-treated control cells as determined by a one-way ANOVA with Dunnett's multiple comparison test for each cell line). Specifically, 25 μM cisplatin resulted in a 11.17±2.79-fold increase in pH2AX staining in A549 cells and a 14.45 20±1.82-fold increase in H1299 cells. In contrast, in A549 cells, increasing concentrations of artesuante, when compared to 0.1% DMSO-treated controls, did not cause significantly increased DNA damage (5 μM: 1.19±0.05, 10 μM: 1.35±0.01, 50 μM: 1.71±0.02, 100 μM: 3.27±0.13), even at the highest concentration of artesunate. Alternatively, in the artesunate sensitive H1299 cells, significant DNA damage (p<0.05) was observed following treatment with as little as 10 μM artesunate. Specifically, 5 μM artesunate resulted in a 3.14±0.09-fold increase in pH2AX staining, 10 μM artesunate produced an increase of 4.47±0.26 fold, 50 μM produced an increase of 8.25±1.08, and 100 μM artesunate increased pH2AX staining by 17.97±0.79-fold compared to 0.1% DMSO control-treated cells.

2.10 Dependence of Artesunate Sensitivity on KEAP1 in NSCLC

A549 cells harbor a G333C mutation in KEAP1, while both H1299 and H1563 cells are wild type for KEAP1 and NFE2L2 (the gene encoding NRF2), which could account for the higher resistance of A549 cells to artesunate. Because artesunate is known to increase cellular ROS and the KEAP1/NRF2 pathway is a master regulator of cellular response to ROS, it was first assessed whether treatment of A549 (resistant) or H1299 (sensitive) cells with artesuante altered the protein expression of either KEAP1 or NQO-1. NQO-1 is a well established transcriptional target of NRF2 as is used as a proxy to assess NRF2 transcriptional activity by Western blot. Cells were treated with 10 μm artesunate for 0, 6, or 24 h prior to lysate collection and a Western blot for KEAP1, NQO-1, and B-ACTIN was performed. The KEAP1 antibody used for Western blotting was generated using a peptide present in both the wild-type and G333C mutant KEAP1 and therefore is able to detect both the wild-type and mutant forms from the cell lines tested. As seen in FIG. 12, A549 cells had reduced basal expression of KEAP1 and increased basal expression of NQO-1 compared to H1299 cells, which is as expected since A549 cells harbor an inactivation mutation in KEAP1. Treatment of both cell lines with artesunate for 24 h resulted in decreased expression of KEAP1. Additionally, in H1299 cells, a slight but detectable increase in NQO-1 protein is observed after 24 h treatment with 10 μM artesunate.

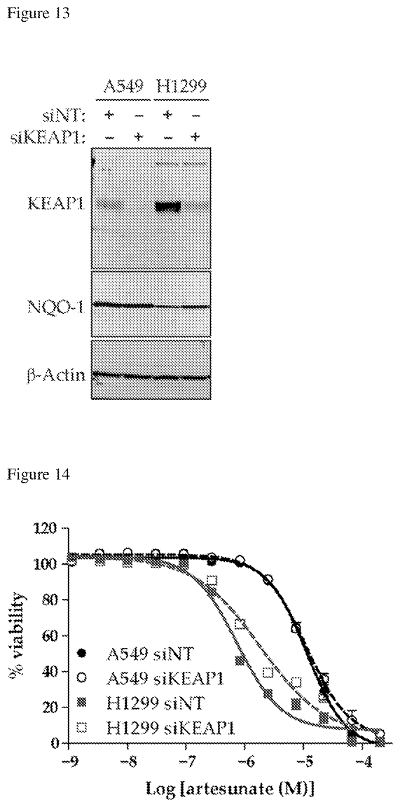

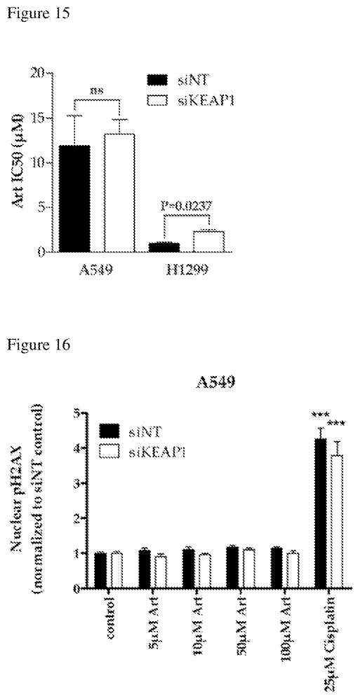

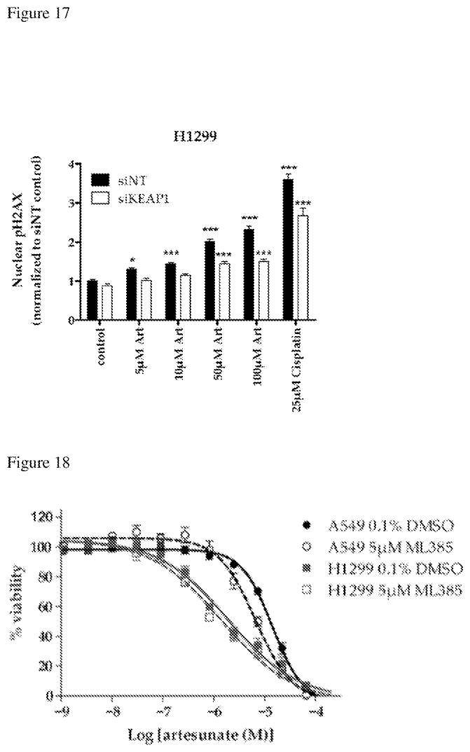

Since KEAP1 expression is reduced by artesunate in both cell lines, to determine whether KEAP1 expression regulates sensitivity to artesunate, we used siRNA to knockdown KEAP1 in both H1299 (sensitive) and A549 (resistant) cells. The siRNA used for this experiment was a pool of four sequences that did not overlap with the mutation site found in A549 cells; therefore, they were predicted to knockdown both wild-type and mutant KEAP1 mRNA and thus reduce KEAP1 protein levels. Knocking down mutant KEAP1 in A549 cells did not affect NQO-1 expression or response to artesunate (FIG. 13). However, knocking down KEAP1 in H1299 cells resulted in increased NQO-1 expression, indicating activation of the NRF2 antioxidant transcriptional pathway. Additionally, knocking down KEAP1 in H1299 cells increased the mean IC50 of artesunate from 0.98 μM in siNT-transfected cells to 2.30 μM in siKEAP1-transfected cells, (FIGS. 14 and 15). Next the effect of knocking down KEAP1 on artesuante induced DNA damage was assessed. In cells with an inactivating KEAP1 mutation, A549, knocking down KEAP1 had no effect on artesuante induced DNA damage. In these cells, only treatment with 25 μM cisplatin resulted in a significant increase in DNA damage compared to matched control cells with a fold change in pH2AX staining of 4.255±0.759 in siNT-transfected cells and 3.791±0.957 in siKEAP1-transfected cells (see FIG. 16). In H1299 cells, knocking down KEAP1 resulted in a significant decrease in artesunate induced DNA damage compared to cells treated with non-targeting control siRNA (siNT) when cells are treated with 50 μM and 100 μM artesunate or with 25 μM cisplatin (FIG. 17). Specifically, 50 μM artesunate resulted in a 2.203±0.170-fold increase in DNA damage in siNT-transfected cells while only a 1.440±0.186-fold increase in DNA damage was observed in siKEAP1-transfected cells when compared to matched control-treated cells. Treatment with 100 μM artesunate and 25 μM cisplatin also resulted in significantly less DNA damage in KEAP1 knockdown cells with 1.502±0.168- and 2.678±0.580-fold nuclear pH2AX staining, respectively compared to 2.238±0.251 and 3.599±0.414 for siNT-transfected cells. Therefore, dysregulation of the NRF2 pathway through decreased expression of KEAP1 or loss of function KEAP1 mutations causes increased resistance to artesunate in NSCLC cell lines.

2.11 NRF2 Inhibition to Sensitize Resistant NSCLC Cells (KEAP1 Mutant) to Artesunate

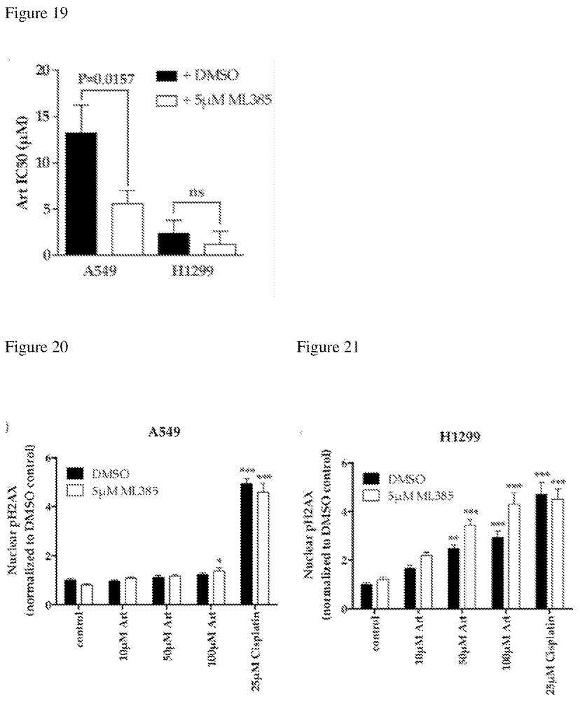

Since activation of the NRF2 pathway occurs downstream of KEAP1 loss, it was next investigated whether the pharmacological inhibition of NRF2 could sensitize resistant A549 cells to artesunate. ML385 (N-[4-[2,3-Dihydro-1-(2-methylbenzoyl)-1H-indol-5-yl]-5-methyl-2-thiazolyl]-1,3-benzodioxole-5-acetamide) was used, a small molecule shown to bind NRF2 and inhibit its function as a transcription factor by preventing DNA binding, to test the effect of NRF2 inhibition on artesunate sensitivity in both A549 (less sensitive) and H1299 (sensitive) cells. Prior to treating cells with a combination of artesuante and ML385 we first assessed the effect of ML385 alone on A549 and H1299 cells. ML385 had minimal effect on cell viability as a single agent in either cell line and there was no significant difference in IC50 observed between the two cell lines tested. Next A549 and H1299 cells were treated with increasing concentrations of artesunate alone or in combination with 5 μM ML385. In order to control for any effect of the addition of 5 μM ML385 had on the cells directly the percent viability of cells treated with artesunate alone was normalized to a DMSO (vehicle) control while cells treated with artesunate+5 μM ML385 were normalized to cells treated with 5 μM ML385. The addition of 5 μM ML385 caused the IC50 to shift from 13.3 μM in A549 cells treated with artesunate plus 0.1% DMSO to 5.60 μM in cells treated with artesunate plus 5 μM ML385 indicating that NRF2 inhibition sensitized these cells to artesunate, (see FIGS. 18 and 19). In the H1299, artesunate sensitive, cell line there was also a slight leftward shift in IC50 (2.35 μM to 1.19 μM in combination with ML385); however, this change was not statistically significant, (see FIG. 19). Next the ability of 5 μM ML385 to enhance artesunate induced DNA damage in A549 and H1299 cells was assessed. For this assay, cells were pretreated for 24 h with 5 μM ML385 or 0.03% DMSO. The next day, fresh media was prepared with 0.03% DMSO or 5 μM ML385 with alone or with 10 μM, 50 μM, or 100 μM artesunate or 25 μM cisplatin and cells were incubated for an additional 24 h. Nuclear pH2AX staining was quantified from three independent experiments and was normalized to control cells which were pretreated with 0.03% DMSO (FIGS. 20 and 21). ML385 alone did not increase DNA damage in A549 (resistant) or H1299 (sensitive) cells. Cotreatment with ML385 and artesunate resulted in statistically significant increase in DNA damage in A549 (less sensitive) cells treated only at the highest dose of 100 μM artesunate when compared to cells treated with 5 μM ML385 alone. Specifically 100 μM artesunate plus 5 μM ML385 resulted in 1.390±0.327-fold DNA damage compared to 0.806±0.142 in cells treated with 5 μM ML385 alone. In H1299 cells, cotreatment with artesunate and ML385 resulted in significant increases in DNA damage with both 50 μM and 100 μM artesunate (3.431±0.685 and 4.309±1.309, respectively), but not in cells treated with 10 μM artesunate (2.187±0.401).

2.12 Artesunate and NRF2 Inhibition are Synergistic

To better understand the effectiveness of combining artesunate with the NRF2 inhibitor, ML385, it was next tested whether this drug combination was synergistic. A synergistic drug interaction is when one or both of the drugs used enhances the effectiveness of the partner drug; therefore, allowing lower concentrations of each drug to be used to achieve a clinical benefit in patients. To assess synergy, we utilized a 6×6 checkerboard method coupled with both Bliss and ZIP models to assess drug combinations. The Bliss independence model has been one of the standards for assessing drug combinations since it was introduced by in 1933 (Bliss, C. I. The Toxicity of Poisons Applied Jointly1. Ann. Appl. Biol. 1939, 26, 585-615, doi:10.1111/j.1744-7348.1939.tb06990.x), while the ZIP model was developed in 2015 to address some of the limitations of the Loewe and Bliss models (Yadav, B.; Wennerberg, K.; Aittokallio, T.; Tang, J. Searching for Drug Synergy in Complex Dose-Response Landscapes Using an Interaction Potency Model. Comput. Struct. Biotechnol. J. 2015, 13, 504-513, doi:10.1016/j.csbj.2015.09.001).

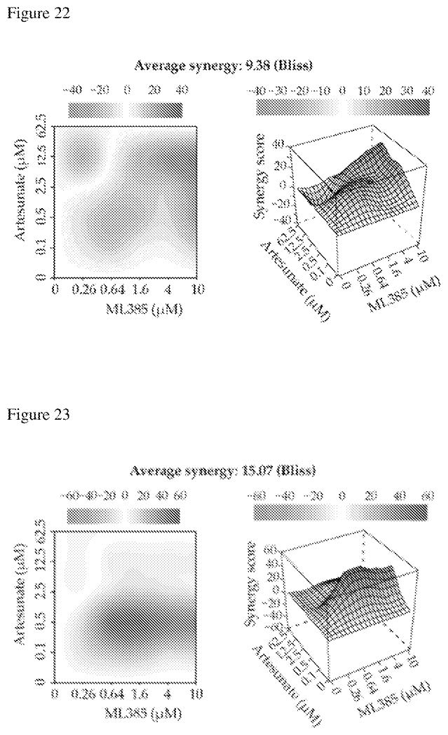

The output of both of these models is a synergy score in which a negative score indicates antagonism while a positive score indicates synergy. When artesunate and ML385 were combined in A549 (artesunate resistant) cells, the Bliss score was 9.38 (FIG. 22), while the ZIP score was 8.744. In the H1299 (artesunate sensitive), the Bliss score was 15.07 (FIG. 23), and the ZIP score was 16.593. This shows that artesunate and ML385 are synergistic in NSCLC cell lines independent of KEAP1 mutational status. In addition, synergy is observed at clinically achievable plasma concentrations (0.1-0.5 μM) concentrations of artesunate.

1. An anti-cancer agent comprising at least one compound having at least one 1,2,4-trioxane moiety or a combination of
a) at least one compound having at least one 1,2,4-trioxane moiety and
b) at least one chlorogenic acid.

2. The anti-cancer agent according to claim 1, wherein the compounds comprising at least one 1,2,4-trioxane moiety are selected from those of formulae (I) to (V) 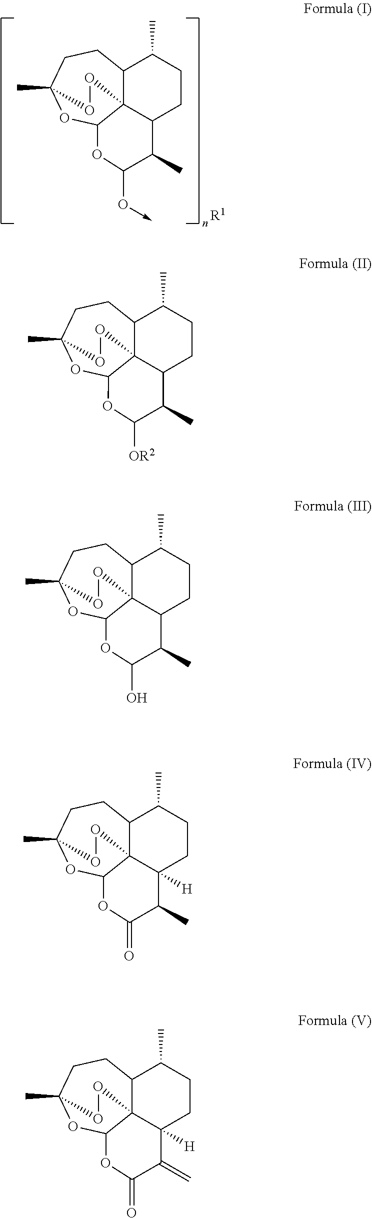
and, where applicable, pharmaceutically acceptable salts of the aforementioned compounds of formulae (I) and (II) wherein
in formula (I)
the arrow denotes the bond between the depicted oxygen atom to the residue R¹
n is an integer of more than 1, preferably 2 to 10, more preferably 2, 3 or 4 and even more preferably 2 or 3
R¹ is a residue that is n times substitued by the residue depicted in the rounded bracket, and is preferably C₁-C₁₈-alkyl or C₂-C₁₈-alkenyl or —(CO)ₙ(R³), wherein the carboxyl groups together with the oxygen bound to the residue R¹ form a carboxylic ester moiety and R³ is C₁-C₁₈-alkane-n-yl or C₂-C₁₈-alkene-n-yl
whereby
the aforementioned C₁-C₁₈-alkyl, C₂-C₁₈-alkenyl, C₁-C₁₈-alkane-n-yl, C₂-C₁₈-alkene-n-yl groups are
either not, once, twice, or more than twice interrupted by non-successive functional groups selected from the group consisting of:
—O—, —S—, —SO₂—, —SO—, —SO₂NR⁴—, NR⁴SO₂—, —NR⁴—, —CO—, —O(CO)—, (CO)O—, —O(CO)O—, —NR⁴(CO)NR⁴—, NR⁴(CO)—, —(CO)NR⁴—, —NR⁴(CO)O—, —O(CO)NR⁴—,
and
either not, additionally, or alternatively either once, twice or more than twice interrupted by bivalent residues selected from the group consisting of heterocyclo-diyl, and aryldiyl,
and
either not, additionally, or alternatively either once, twice or more than twice substituted by substituents selected from the group consisting of:
hydroxy, halogen, cyano, azido, C₆-C₁₄-aryl, C₁-C₈-alkoxy, C₁-C₈-alkylthio, —SO₃M, —COOM, —PO₃M₂, —PO(N(R⁵)₂)₂, —PO(OR⁵)₂, —SO₂N(R⁴), —N(R⁴)₂, —CO₂N(R⁵)₂, —COR⁴, —OCOR⁴, —NR⁴(CO)R⁵, —(CO)OR⁴, —NR⁴(CO)N(R⁴)₂
And in formula (II)
R² is C₁-C₁₈-alkyl or C₂-C₁₈-alkenyl or —(CO)R³, wherein the carboxyl groups together with the oxygen bound to the residue R¹ form a carboxylic ester moiety and R³ is C₁-C₁₈-alkyl or C₂-C₁₈-alkenyl
whereby
the aforementioned C₁-C₁₈-alkyl and C₂-C₁₈-alkenyl groups are
either not, once, twice, or more than twice interrupted by non-successive functional groups selected from the group consisting of:
—O—, —S—, —SO₂—, —SO—, —SO₂NR⁴—, —NR⁴SO₂—, —NR⁴, —CO—, —O(CO)—, —(CO)O—, —O(CO)O—, —NR⁴(CO)NR⁴—, NR⁴(CO)—, —(CO)NR⁴—, —NR⁴(CO)O— or —O(CO)NR⁴—
and
either not, additionally, or alternatively either once, twice or more than twice interrupted by bivalent residues selected from the group consisting of heterocyclo-diyl, and aryldiyl,
and
either not, additionally, or alternatively either once, twice or more than twice substituted by substituents selected from the group consisting of:
hydroxy, halogen, cyano, azido, C₆-C₁₄-aryl, C₁-C₈-alkoxy, C₁-C₈-alkylthio, —SO₃M, —COOM, PO₃M₂, —PO(N(R⁵)₂)₂, PO(OR⁵)₂, —SO₂N(R⁴)₂, —N(R⁴)₂, —CO₂N(R⁵)₂, —COR⁴, —OCOR⁴, —NR⁴(CO)R⁵, —(CO)OR⁴ or —NR⁴(CO)N(R⁴)₂
whereby in all formulae above where used
R⁴ is independently selected from the group consisting of hydrogen, C₁-C₈-alkyl, C₆-C₁₄-aryl, and heterocyclic or —N(R⁴)₂ as a whole is a N-containing heterocycle,
R⁵ is independently selected from the group consisting of C₁-C₈-alkyl, C₆-C₁₄-aryl, and heterocyclic or —N(R⁵)₂ as a whole is a N-containing heterocycle and
M is hydrogen, or 1/q equivalent of an q-valent metal ion or is an ammonium ion or a guanidinium ion or a primary, secondary, tertiary or quarternary organic ammonium ion, in particular those of formula [N(C₁-C₁₈-alkyl)ₛHₜ]⁺ wherein s is 1,2,3 or 4 and t is (4-s).

3. The anti-cancer agent according to claim 1 wherein the compounds comprising at least one 1,2,4-trioxane moiety are selected from those of formula (IIa), artemether, and of formula (IIb), artesunate, and pharmaceutically acceptable salts of artesunate.

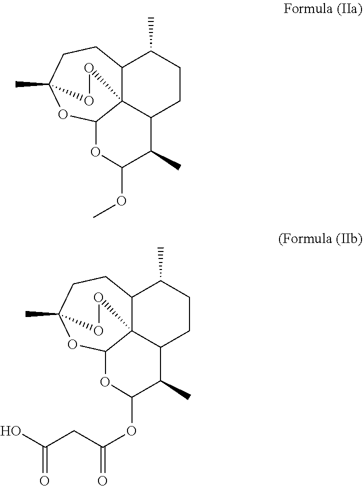

4. The anti-cancer agent according to claim 1, wherein the compounds comprising at least one 1,2,4-trioxane moiety are selected from artesunate and pharmaceutically acceptable salts of artesunate

5. The anti-cancer agent according to claim 1, wherein the at least one chlorogenic acid is selected from 3-O-caffeoylquinic acid, 4-O-caffeoylquinic acid, 5-O-caffeoylquinic acid, 3-O-ferruoylquinic acid, 4-O-ferruoylquinic acid, 5-O-ferruoylquinic acid, 3,4-dicaffeoylquinic acid, 3,5-dicaffeoylquinic acid and 4,5-dicaffeoylquinic acid.

6. The anti-cancer agent according to claim 1, wherein the molar ratio between the compound or the compounds having at least one 1,2,4-trioxane moiety and the chlorogenic acid or chlorogenic acids present in the combination is between 2 and 0.002.

7. The anti-cancer agent according to claim 1, wherein the compounds comprising at least one 1,2,4-trioxane moiety are obtained via extraction of Artemisia annua.

8. The anti-cancer agent according to claim 1, comprising extracts of Artemisia annua.

9. The anti-cancer agent according to claim 1, wherein the at least one chlorogenic acid is obtained via extraction of coffee or tea.

10. (canceled)

11. (canceled)

12. (canceled)

13. The anti-cancer agent according to claim 1, wherein the molar ratio between the compound or the compounds having at least one 1,2,4-trioxane moiety and the chlorogenic acid or chlorogenic acids present in the combination is between 2 and 0.002.

14. (canceled)

15. Pharmaceutical compositions comprising an anti-cancer agent according to claim 1.

16. Pharmaceutical compositions comprising an anti-cancer agent according to claim 1 further comprising at least one additional therapeutic agent.

17. Pharmaceutical compositions comprising an anti-cancer agent according to claim 16 wherein the additional therapeutic agents are selected from the group of gemtricitabine, cisplatin, carboplatin, pemetrexed or paclitaxel and/or a NRF2 inhibitor such as ML385.

18. Kit comprising:
(a) a dosage of an anticancer-agent or pharmaceutical composition according to claim 1 and
(b) a dosage of an additional therapeutic agent being a platinum-based doublet chemotherapy (PT-DC) and
(c) instructions for using the anti-cancer agent or pharmaceutical composition according to claim 1 and the additional therapeutic agent.

19. (canceled)

20. The anti-cancer agent according to claim 1 for use as a medicament for the treatment and/or prevention of cancer or for the prolongation of a subject having cancer.

21. A method of preventing or treating cancer in a subject having cancer or for prolonging survival of a subject having cancer comprising administering therapeutically effective amounts of the anti-cancer agent according to claim 1.

22. (canceled)

23. The method according to claim 21, wherein the dose range of the anti-cancer agents or pharmaceutical compositions per day is from 0.01 to 100 mg/kg of body weight, preferably from 0.1 to 50 mg/kg of body weight, calculated on the sum of compounds having at least one 1,2,4-trioxane moiety.

24. (canceled)

25. The method according to claim 21 comprising administering to a subject in need thereof at least
(a) the anti-cancer agent
(b) an additional therapeutic agent selected from the group consisting of: a platinum-based doublet chemotherapy (PT-DC), wherein the PT-DC is a combination of (i) gemcitabine and cisplatin, (ii) pemetrexed and cisplatin, or (iii) paclitaxel and carboplatin.

26. The method according to claim 25, wherein the additional therapeutic agent is a combination of paclitaxel and carboplatin.

27. (canceled)

28. (canceled)

29. (canceled)

30. (canceled)

31. A method for destructing or inhibiting the growth of cancer cells comprising exposing the cancer cells to an effective amount of the anti-cancer agents, pharmaceutical compositions or kits according to claim 1 in vitro or in vivo.

32. (canceled)
